(19) United States
(12) Patent Application Publication
(10) Pub. No.: US 2025/0221968 A1
(43) Pub. Date: Jul. 10, 2025

(54) TREATMENTS AND METHODS FOR INCREASING DOPAMINE

(71) Applicant: Oxford University Innovation Limited, Oxford (GB)

(72) Inventors: Yanfeng Zhang, Oxford (GB); Stephanie J. Cragg, Oxford (GB)

(21) Appl. No.: 18/846,959

(22) PCT Filed: Mar. 17, 2023
(86) PCT No.: PCT/GB23/50660

§ 371 Date: Sep. 13, 2024

(30) Foreign Application Priority Data
Mar. 17, 2022 (GB) 2203728.7

(51) Int. Cl. A61K 31/437; A61K 31/13; A61P 25/16

(52) U.S. Cl. CPC A61K 31/437; A61K 31/13; A61P 25/16

(57) ABSTRACT

The present invention relates to methods for increasing dopamine levels in a subject. The present invention also relates to the treatment of condition or disorder in which reduced levels of dopamine are implicated, and agents for use in such treatments.

The present invention relates to treatments and methods for increasing dopamine levels in a subject, especially in the central nervous system (CNS). The present invention also relates to the treatment of conditions or disorders in which reduced levels of dopamine are implicated, and agents for use in such treatments.

### BACKGROUND

Striatal cholinergic interneurons (ChIs) acting at nicotinic acetylcholine receptors (nAChRs) on dopamine axons are thought to promote dopamine output. Synchronised activity in Chis can drive dopamine release (Cachope, R. et al. Selective activation of cholinergic interneurons enhances accumbal phasic dopamine release: setting the tone for reward processing. Cell Rep 2, 33-41, doi: 10.1016/j.celrep.2012.05.011 (2012) and Threlfell, S. et al. Striatal dopamine release is triggered by synchronized activity in cholinergic interneurons. Neuron 75, 58-64, doi: 10.1016/j.neuron.2012.04.038 (2012)), while ChI pauses enhance dopamine release during burst-like high frequencies of dopamine neuron activity (Cragg, S. J. Meaningful silences: how dopamine listens to the ACh pause. Trends Neurosci 29, 125-131, doi: 10.1016/j.tins.2006.01.003 (2006) and Rice, M. E. & Cragg, S. J. Nicotine amplifies reward-related dopamine signals in striatum. Nat Neurosci 7, 583-584, doi: 10.1038/nn1244 (2004)). However, an unexplained and critical observation is that dopamine release can be substantially lower when nAChRs are activated than inactivated (Rice & Cragg, 2004), questioning whether ChIs promote or attenuate dopamine output.

Lowered levels of dopamine have been associated with a number of conditions, including addiction, Alzheimer's disease, attention deficit hyperactivity disorder (ADHD), bipolar disorder, dopa-responsive dystonia and DRD-plus, Huntington's disease, multiple sclerosis, obsessive compulsive disorder (OCD), Parkinson's disease (including resting tremor in Parkinson's disease), schizophrenia, tics (i.e. a repetitive involuntary movement or sound) and Tourette's syndrome.

Traditional treatment for diseases such as Parkinson's disease, such as levodopa, are thought to provide a general elevation of dopamine level in the striatum, including through release from inappropriate non-dopaminergic inputs. This can lead to the development of profound side-effects after a few years. Therefore, alternative therapeutic approaches are required to reduce the risk of such side effects.

Parkinson's disease is the second most common neurodegenerative disorder. The core motor symptoms are caused by the loss of midbrain dopamine (DA) neurons. Levodopa, a DA precursor, provides the most effective symptomatic treatment by elevating DA release from residual neurons. However, chronic levodopa administration at concentrations sufficient to achieve symptomatic relief, particularly in late stage PD patients or those on long-term levodopa treatment, leads to the emergence of disabling motor side effects, termed levodopa-induced dyskinesia (LID).

### BRIEF SUMMARY OF THE DISCLOSURE

The present inventors have revealed that striatal cholinergic interneurons can powerfully inhibit dopamine release in the striatum after activating nicotinic acetylcholine receptors (nAChRs) on dopamine axons. Administration of a nAChR antagonist/blocker is expected to relieve an inhibition of dopamine release caused by striatal cholinergic interneurons, thereby providing an effective treatment for a condition or disorder in which reduced levels of dopamine are implicated. Thus, the present invention targets the reduced levels of dopamine seen in a number of conditions.

The present invention resides in the recognition that therapy involving the administration of a nicotinic acetylcholine receptor (nAChR) antagonist/blocker, or a pharmaceutically acceptable salt thereof, results in an increase in the level of dopamine in the CNS. The administration of a nAChR antagonist/blocker itself targets the inhibition of dopamine release caused by striatal cholinergic interneurons acting at the nAChR.

Herein, the term “nicotinic acetylcholine receptor antagonist/blocker” (also known as nicotinic antagonist/blocker), refers to a compound which inhibits the action of acetylcholine (ACh) at nicotinic acetylcholine receptors. The term “antagonist/blocker” encompasses competitive antagonists and non-competitive antagonists.

### Therapeutic Uses and Applications

In a first aspect of the invention there is provided a nicotinic acetylcholine receptor (nAChR) antagonist/blocker, or a pharmaceutically acceptable salt thereof, for use in the treatment of a condition or disorder in which reduced levels of dopamine are implicated.

In another aspect of the invention there is provided a nicotinic acetylcholine receptor (nAChR) antagonist/blocker, or a pharmaceutically acceptable salt thereof, for use in the treatment of a condition or disorder in which reduced levels of dopamine in the central nervous system (CNS) are implicated.

In another aspect of the invention there is provided a nicotinic acetylcholine receptor (nAChR) antagonist/blocker, or a pharmaceutically acceptable salt thereof, for use in the treatment of a condition or disorder in which reduced levels of dopamine in the brain are implicated.

In another aspect of the invention there is provided a nicotinic acetylcholine receptor (nAChR) antagonist/blocker, or a pharmaceutically acceptable salt thereof, for use in the treatment of a condition or disorder in which reduced levels of dopamine in the striatum are implicated.

In another aspect of the invention there is provided a nicotinic acetylcholine receptor (nAChR) antagonist/blocker, or a pharmaceutically acceptable salt thereof, for use in the treatment of a condition or disorder in which reduced levels of dopamine in the dorsal striatum, ventral striatum, ventral tegmental area, basal ganglia and/or the substantia nigra are implicated.

In another aspect of the invention there is provided a nicotinic acetylcholine receptor (nAChR) antagonist/blocker, or a pharmaceutically acceptable salt thereof, for use in the treatment of a condition or disorder in which reduced levels of dopamine in the dorsal striatum (including the caudate and putamen) are implicated.

In further aspect of the invention there is provided a nicotinic acetylcholine receptor (nAChR) antagonist/blocker, or a pharmaceutically acceptable salt thereof, for use in increasing the level of dopamine in the CNS.

In another aspect of the invention there is provided a nicotinic acetylcholine receptor (nAChR) antagonist/blocker, or a pharmaceutically acceptable salt thereof, for use in increasing the level of dopamine in the brain.

In another aspect of the invention there is provided a nicotinic acetylcholine receptor (nAChR) antagonist/blocker, or a pharmaceutically acceptable salt thereof, for use in increasing the level of dopamine in the striatum.

In another aspect of the invention there is provided a nicotinic acetylcholine receptor (nAChR) antagonist/blocker, or a pharmaceutically acceptable salt thereof, for use in increasing the level of dopamine in the dorsal striatum, ventral striatum, ventral tegmental area, basal ganglia, and/or the substantia nigra.

In another aspect of the invention there is provided a nicotinic acetylcholine receptor (nAChR) antagonist/blocker, or a pharmaceutically acceptable salt thereof, for use in increasing the level of dopamine in the dorsal striatum (including the caudate and putamen).

In another aspect of the invention there is provided a nicotinic acetylcholine receptor (nAChR) antagonist/blocker, or a pharmaceutically acceptable salt thereof, for use in increasing the level of dopamine in the CNS of a subject in the treatment of a condition or disorder in which reduced levels of dopamine in the CNS are implicated.

In another aspect of the invention there is provided a nicotinic acetylcholine receptor (nAChR) antagonist/blocker, or a pharmaceutically acceptable salt thereof, for use in increasing the level of dopamine in the brain of a subject in the treatment of a condition or disorder in which reduced levels of dopamine in the brain are implicated.

In another aspect of the invention there is provided a nicotinic acetylcholine receptor (nAChR) antagonist/blocker, or a pharmaceutically acceptable salt thereof, for use in increasing the level of dopamine in the striatum of a subject in the treatment of a condition or disorder in which reduced levels of dopamine in the striatum are implicated.

In another aspect of the invention there is provided a nicotinic acetylcholine receptor (nAChR) antagonist/blocker, or a pharmaceutically acceptable salt thereof, for use in increasing the level of dopamine in the dorsal striatum, ventral striatum, ventral tegmental area, basal ganglia and/or the substantia nigra of a subject, in the treatment of a condition or disorder in which reduced levels of dopamine in the dorsal striatum, ventral striatum, ventral tegmental area, basal ganglia and/or the substantia nigra are implicated.

In another aspect of the invention there is provided a nicotinic acetylcholine receptor (nAChR) antagonist/blocker, or a pharmaceutically acceptable salt thereof, for use in increasing the level of dopamine in the dorsal striatum (including the caudate and putamen) of a subject in the treatment of a condition or disorder in which reduced levels of dopamine in the dorsal striatum (including the caudate and putamen) are implicated.

In another aspect of the invention, there is provided the use of a nicotinic acetylcholine receptor (nAChR) antagonist/blocker, or a pharmaceutically acceptable salt thereof, in the manufacture of a medicament for use in treatment of a subject suffering from a disorder associated with reduced levels of dopamine in the CNS.

In another aspect of the invention, there is provided the use of a nicotinic acetylcholine receptor (nAChR) antagonist/blocker, or a pharmaceutically acceptable salt thereof, in the manufacture of a medicament for use in treatment of a subject suffering from a disorder associated with reduced levels of dopamine in the brain.

In another aspect of the invention, there is provided the use of a nicotinic acetylcholine receptor (nAChR) antagonist/blocker, or a pharmaceutically acceptable salt thereof, in the manufacture of a medicament for use in treatment of a subject suffering from a disorder associated with reduced levels of dopamine in the striatum.

In another aspect of the invention, there is provided the use of a nicotinic acetylcholine receptor (nAChR) antagonist/blocker, or a pharmaceutically acceptable salt thereof, in the manufacture of a medicament for use in treatment of a subject suffering from a disorder associated with reduced levels of dopamine in the dorsal striatum, ventral striatum, ventral tegmental area, basal ganglia and/or the substantia nigra.

In another aspect of the invention, there is provided the use of a nicotinic acetylcholine receptor (nAChR) antagonist/blocker, or a pharmaceutically acceptable salt thereof, in the manufacture of a medicament for use in treatment of a subject suffering from a disorder associated with reduced levels of dopamine in the dorsal striatum (including the caudate and putamen).

In another aspect of the invention there is provided the use of a nicotinic acetylcholine receptor (nAChR) antagonist/blocker, or a pharmaceutically acceptable salt thereof, in the manufacture of a medicament for use in increasing the level of dopamine in the CNS.

In another aspect of the invention there is provided the use of a nicotinic acetylcholine receptor (nAChR) antagonist/blocker, or a pharmaceutically acceptable salt thereof, in the manufacture of a medicament for use in increasing the level of dopamine in the brain.

In another aspect of the invention there is provided the use of a nicotinic acetylcholine receptor (nAChR) antagonist/blocker, or a pharmaceutically acceptable salt thereof, in the manufacture of a medicament for use in increasing the level of dopamine in the striatum.

In another aspect of the invention there is provided the use of a nicotinic acetylcholine receptor (nAChR) antagonist/blocker, or a pharmaceutically acceptable salt thereof, in the manufacture of a medicament for use in increasing the level of dopamine in the dorsal striatum, ventral striatum, ventral tegmental area, basal ganglia and/or the substantia nigra.

In another aspect of the invention there is provided the use of a nicotinic acetylcholine receptor (nAChR) antagonist/blocker, or a pharmaceutically acceptable salt thereof, in the manufacture of a medicament for use in increasing the level of dopamine in the dorsal striatum (including the caudate and putamen).

In another aspect of the invention, there is provided the use of a nicotinic acetylcholine receptor (nAChR) antagonist/blocker, or a pharmaceutically acceptable salt thereof, in the manufacture of a medicament for use in increasing the level of dopamine in the CNS, in the treatment of a subject suffering from a disorder associated with reduced levels of dopamine in the CNS.

In another aspect of the invention, there is provided the use of a nicotinic acetylcholine receptor (nAChR) antagonist/blocker, or a pharmaceutically acceptable salt thereof, in the manufacture of a medicament for use in increasing the level of dopamine in the brain, in the treatment of a subject suffering from a disorder associated with reduced levels of dopamine in the brain.

In another aspect of the invention, there is provided the use of a nicotinic acetylcholine receptor (nAChR) antagonist/blocker, or a pharmaceutically acceptable salt thereof, in the manufacture of a medicament for use in increasing the level of dopamine in the CNS, in the treatment of a subject suffering from a disorder associated with reduced levels of dopamine in the CNS.

In another aspect of the invention, there is provided the use of a nicotinic acetylcholine receptor (nAChR) antagonist/blocker, or a pharmaceutically acceptable salt thereof, in the manufacture of a medicament for use in increasing the level of dopamine in the striatum, in the treatment of a subject suffering from a disorder associated with reduced levels of dopamine in the striatum.

In another aspect of the invention, there is provided the use of a nicotinic acetylcholine receptor (nAChR) antagonist/blocker, or a pharmaceutically acceptable salt thereof, in the manufacture of a medicament for use in increasing the level of dopamine in the dorsal striatum, ventral striatum, ventral tegmental area, basal ganglia and/or the substantia nigra, in the treatment of a subject suffering from a disorder associated with reduced levels of dopamine in the dorsal striatum, ventral striatum, ventral tegmental area, basal ganglia and/or the substantia nigra.

In another aspect of the invention, there is provided the use of a nicotinic acetylcholine receptor (nAChR) antagonist/blocker, or a pharmaceutically acceptable salt thereof, in the manufacture of a medicament for use in increasing the level of dopamine in the dorsal striatum (including the caudate and putamen), in the treatment of a subject suffering from a disorder associated with reduced levels of dopamine in the dorsal striatum (including the caudate and putamen).

In another aspect of the invention, there is provided the use of a nAChR antagonist/blocker, or a pharmaceutically acceptable salt thereof, for the manufacture of a medicament for increasing the level of dopamine in the CNS in the treatment of a condition or disorder in which reduced levels of dopamine in the CNS are implicated.

In another aspect of the invention, there is provided the use of a nAChR antagonist/blocker, or a pharmaceutically acceptable salt thereof, for the manufacture of a medicament for increasing the level of dopamine in the brain in the treatment of a condition or disorder in which reduced levels of dopamine in the brain are implicated.

In another aspect of the invention, there is provided the use of a nAChR antagonist/blocker, or a pharmaceutically acceptable salt thereof, for the manufacture of a medicament for increasing the level of dopamine in the striatum in the treatment of a condition or disorder in which reduced levels of dopamine in the striatum are implicated.

In another aspect of the invention, there is provided the use of a nAChR antagonist/blocker, or a pharmaceutically acceptable salt thereof, for the manufacture of a medicament for increasing the level of dopamine in the dorsal striatum, ventral striatum, ventral tegmental area, basal ganglia and/or the substantia nigra in the treatment of a condition or disorder in which reduced levels of dopamine in the dorsal striatum, ventral striatum, ventral tegmental area, basal ganglia and/or the substantia nigra are implicated.

In another aspect of the invention, there is provided the use of a nAChR antagonist/blocker, or a pharmaceutically acceptable salt thereof, for the manufacture of a medicament for increasing the level of dopamine in the dorsal striatum (including the caudate and putamen), in the treatment of a condition or disorder in which reduced levels of dopamine in the dorsal striatum (including the caudate and putamen) are implicated.

### Methods of Treatment

In another aspect of the invention there is provided a method of treating a condition or disorder in which reduced levels of dopamine in the CNS are implicated, said method comprising administering to a subject in need thereof a therapeutically effective amount of a nicotinic acetylcholine receptor (nAChR) antagonist/blocker, or a pharmaceutically acceptable salt thereof.

In another aspect of the invention there is provided a method of treating a condition or disorder in which reduced levels of dopamine in the brain are implicated, said method comprising administering to a subject in need thereof a therapeutically effective amount of a nicotinic acetylcholine receptor (nAChR) antagonist/blocker, or a pharmaceutically acceptable salt thereof.

In another aspect of the invention there is provided a method of treating a condition or disorder in which reduced levels of dopamine in the striatum are implicated, said method comprising administering to a subject in need thereof a therapeutically effective amount of a nicotinic acetylcholine receptor (nAChR) antagonist/blocker, or a pharmaceutically acceptable salt thereof.

In another aspect of the invention there is provided a method of treating a condition or disorder in which reduced levels of dopamine in the dorsal striatum, ventral striatum, ventral tegmental area, basal ganglia and/or the substantia nigra are implicated, said method comprising administering to a subject in need thereof a therapeutically effective amount of a nicotinic acetylcholine receptor (nAChR) antagonist/blocker, or a pharmaceutically acceptable salt thereof.

In another aspect of the invention there is provided a method of treating a condition or disorder in which reduced levels of dopamine in the dorsal striatum (including the caudate and putamen) are implicated, said method comprising administering to a subject in need thereof a therapeutically effective amount of a nicotinic acetylcholine receptor (nAChR) antagonist/blocker, or a pharmaceutically acceptable salt thereof.

In another aspect of the invention there is provided a method of increasing the level of dopamine in the CNS of a subject, the method comprising administering a therapeutically effective amount of a nicotinic acetylcholine receptor (nAChR) antagonist/blocker, or a pharmaceutically acceptable salt thereof, to the subject.

In another aspect of the invention there is provided a method of increasing the level of dopamine in the brain of a subject, the method comprising administering a therapeutically effective amount of a nicotinic acetylcholine receptor (nAChR) antagonist/blocker, or a pharmaceutically acceptable salt thereof, to the subject.

In another aspect of the invention there is provided a method of increasing the level of dopamine in the striatum of a subject, the method comprising administering a therapeutically effective amount of a nicotinic acetylcholine receptor (nAChR) antagonist/blocker, or a pharmaceutically acceptable salt thereof, to the subject.

In another aspect of the invention there is provided a method of increasing the level of dopamine in the dorsal striatum, ventral striatum, ventral tegmental area, basal ganglia and/or the substantia nigraof a subject, the method comprising administering a therapeutically effective amount of a nicotinic acetylcholine receptor (nAChR) antagonist/blocker, or a pharmaceutically acceptable salt thereof, to the subject.

In another aspect of the invention there is provided a method of increasing the level of dopamine in the dorsal striatum (including the caudate and putamen) of a subject, the method comprising administering a therapeutically effective amount of a nicotinic acetylcholine receptor (nAChR) antagonist/blocker, or a pharmaceutically acceptable salt thereof, to the subject.

In another aspect of the invention, there is provided a method of treating a condition or disorder in which reduced levels of dopamine in the CNS are implicated, said method comprising administering to a subject in need thereof a therapeutically effective amount of a nicotinic acetylcholine receptor (nAChR) antagonist/blocker or a pharmaceutically acceptable salt thereof, wherein administration of nicotinic acetylcholine receptor antagonist/blocker increases the level of dopamine in the CNS.

In another aspect of the invention, there is provided a method of treating a condition or disorder in which reduced levels of dopamine in the brain are implicated, said method comprising administering to a subject in need thereof a therapeutically effective amount of a nicotinic acetylcholine receptor (nAChR) antagonist/blocker or a pharmaceutically acceptable salt thereof, wherein administration of nicotinic acetylcholine receptor antagonist increases the level of dopamine in the brain.

In another aspect of the invention, there is provided a method of treating a condition or disorder in which reduced levels of dopamine in the striatum are implicated, said method comprising administering to a subject in need thereof a therapeutically effective amount of a nicotinic acetylcholine receptor (nAChR) antagonist/blocker or a pharmaceutically acceptable salt thereof, wherein administration of nicotinic acetylcholine receptor antagonist/blocker increases the level of dopamine in the striatum.

In another aspect of the invention, there is provided a method of treating a condition or disorder in which reduced levels of dopamine in the dorsal striatum, ventral striatum, ventral tegmental area, basal ganglia and/or the substantia nigra are implicated, said method comprising administering to a subject in need thereof a therapeutically effective amount of a nicotinic acetylcholine receptor (nAChR) antagonist/blocker or a pharmaceutically acceptable salt thereof, wherein administration of nicotinic acetylcholine receptor antagonist/blocker increases the level of dopamine in the dorsal striatum, ventral striatum, ventral tegmental area, basal ganglia and/or the substantia nigra.

In another aspect of the invention, there is provided a method of treating a condition or disorder in which reduced levels of dopamine in the dorsal striatum (including the caudate and putamen) are implicated, said method comprising administering to a subject in need thereof a therapeutically effective amount of a nicotinic acetylcholine receptor (nAChR) antagonist/blocker or a pharmaceutically acceptable salt thereof, wherein administration of nicotinic acetylcholine receptor antagonist/blocker increases the level of dopamine in the dorsal striatum (including the caudate and putamen).

### Conditions to be Treated

A number of conditions are associated with reduced levels of dopamine. The inventors have demonstrated that administration of a nAChR antagonist/blocker will act to increase the dopamine levels in the CNS (e.g. in the brain, including in the striatum) to treat such conditions. Suitably, the administration of a nAChR antagonist/blocker will act to increase the dopamine levels in the dorsal striatum, ventral striatum, ventral tegmental area, basal ganglia and/or the substantia nigra. Most suitably, the administration of a nAChR antagonist/blocker will act to increase the dopamine levels in the dorsal striatum (including the caudate and putamen).

Suitably, the condition or disorder is selected from addiction (including binge eating, nicotine addiction and alcohol addiction), Alzheimer's disease, attention deficit hyperactivity disorder (ADHD), bipolar disorder, dopa-responsive dystonia and DRD-plus, Huntington's disease, multiple sclerosis, obsessive compulsive disorder (OCD), Parkinson's disease (including resting tremor in Parkinson's disease), schizophrenia, tics (i.e. a repetitive involuntary movement or sound), Tourette's syndrome, depression and reward deficiency syndrome. More suitably, the condition or disorder is selected from Parkinson's Disease or ADHD. Most suitably, the condition or disorder is Parkinson's Disease.

In some situations, the subject may have experienced side effects as a result of taking dopamine precursors (e.g. levodopa) or dopamine agonists to treat a disorder in which reduced levels of dopamine in the CNS are implicated. Suitably, the subject is suffering from levodopa-induced dyskinesia.

Suitably, the subject has reduced levels of dopamine in the CNS, the brain and/or the striatum. More suitably, the subject has reduced levels of dopamine in the dorsal striatum, ventral striatum, ventral tegmental area, basal ganglia and/or the substantia nigra. Most suitably, the subject has reduced levels of dopamine in the dorsal striatum (including the caudate and putamen).

Suitably, the administration of the nicotinic acetylcholine receptor antagonist/blocker promotes dopamine release in the CNS, the brain and/or the striatum. More suitably, the administration of the nicotinic acetylcholine receptor antagonist/blocker promotes dopamine release in the dorsal striatum, ventral striatum, ventral tegmental area, basal ganglia and/or the substantia nigra. Most suitably, the administration of the nicotinic acetylcholine receptor antagonist/blocker promotes dopamine release in the dorsal striatum (including the caudate and putamen).

nAChR Antagonists/Blocker

Suitably, the nAChR antagonist/blocker is selected from Dihydro-β-erythroidine (DHβE), mecamylamine, bPiDDP (1,1′-(1,12-Dodecanediyl)bis[3-methylpyridinium]dibromide), methyllycaconitine, MG 624 (N,N,N-Triethyl-2-[4-(2-phenylethenyl)phenoxy]ethanaminium iodide), SR 16584 (1,3-Dihydro-1-(3-exo)-9-methyl-9-azabicyclo[3.3.1]non-3-yl]-2H-indol-2-one), Catestatin, Chlorisondamine diiodide, 2,2,6,6-tetramethylpiperidin-4-yl heptanoate (TMPH), Chlorisondamine, α-conotoxins (e.g. ACV 1, AulB, El, Iml, MII, PIA, PnlA), A 85380 (3-[(2S)-2-Azetidinylmethoxy]-pyridine, ABT 089 (2-Methyl-3-[(2S)-pyrrolidinylmethoxy]pyridine), ABT 594 ((R)-5-(Azetidin-2-ylmethoxy)-2-chloropyridine), Dianicline ((5aS,8S,10aR)-5a,6,9,10-Tetrahydro-7H, 11H-8,10a-methanopyrido[2′,3′:5,6]pyrano[2,3-d]azepine), RJR 2403 ((E)-N-Methyl-4-(3-pyridinyl)-3-buten-1-amine), TC 2559 (4-(5-ethoxy-3-pyridinyl)-N-methyl-(3E)-3-buten-1-amine), Varenicline (7,8,9,10-Tetrahydro-6,10-methano-6H-pyrazino[2,3-h][3]benzazepine), hexamethonium bromide, tubocurarine chloride, α-bungarotoxin, COG 133, D-amphetamine sulfate, PAMP-20, or a pharmaceutically acceptable salt or derivative thereof.

Suitably, the nAChR antagonist/blocker is selected from Dihydro-β-erythroidine (DHβE), mecamylamine, bPiDDP (1,1′-(1,12-Dodecanediyl)bis[3-methylpyridinium]dibromide), methyllycaconitine, MG 624 (N,N,N-Triethyl-2-[4-(2-phenylethenyl)phenoxy]ethanaminium iodide), SR 16584 (1,3-Dihydro-1-(3-exo)-9-methyl-9-azabicyclo[3.3.1]non-3-yl]-2H-indol-2-one), Catestatin, Chlorisondamine diiodide, 2,2,6,6-tetramethylpiperidin-4-yl heptanoate (TMPH), Chlorisondamine, α-conotoxins (e.g. ACV 1, AulB, El, Iml, MII, PIA, PnlA), A 85380 (3-[(2S)-2-Azetidinylmethoxy]-pyridine, ABT 089 (2-Methyl-3-[(2S)-pyrrolidinylmethoxy]pyridine), ABT 594 ((R)-5-(Azetidin-2-ylmethoxy)-2-chloropyridine), Dianicline ((5aS,8S,10aR)-5a,6,9,10-Tetrahydro-7H, 11H-8,10a-methanopyrido[2′,3′:5,6]pyrano[2,3-d]azepine), RJR 2403 ((E)-N-Methyl-4-(3-pyridinyl)-3-buten-1-amine), TC 2559 (4-(5-ethoxy-3-pyridinyl)-N-methyl-(3E)-3-buten-1-amine), Varenicline (7,8,9,10-Tetrahydro-6,10-methano-6H-pyrazino[2,3-h][3]benzazepine), or a pharmaceutically acceptable salt or derivative thereof.

More suitably, the nicotinic acetylcholine receptor antagonist/blocker is selected from Dihydro-β-erythroidine (DHβE), mecamylamine, bPiDDP, methyllycaconitine, MG 624, SR 16584, Catestatin, Chlorisondamine diiodide, 2,2,6,6-tetramethylpiperidin-4-yl heptanoate (TMPH), Chlorisondamine and α-conotoxins (e.g. ACV 1, AulB, El, Iml, MII, PIA, PnlA), or a pharmaceutically acceptable salt or derivative thereof.

Most suitably, the nicotinic acetylcholine receptor antagonist/blocker is selected from Dihydro-β-erythroidine (DHβE), mecamylamine, Chlorisondamine diiodide, 2,2,6,6-tetramethylpiperidin-4-yl heptanoate (TMPH), Chlorisondamine, or a pharmaceutically acceptable salt or derivative thereof.

The nAChR antagonist/blocker may be a derivative of mecamylamine, for example one or more of the compounds disclosed within WO2013/026852, the entire contents of which are incorporated by reference. The nAChR antagonist/blocker may be a derivative of dihydro-β-erythroidine.

Suitably, the nAChR antagonist/blocker is an antagonist for the α4β2 nAChR receptor. More suitably, the nAChR antagonist/blocker is selective for the α4β2 receptor. It is envisaged that an antagonist/blocker that is selective for the α4β2 receptor will reduce the risk of side effects in the subject to be treated.

Certain α4β2 ligands with antagonistic activity are described in U.S. Pat. Nos. 9,029,557 and 5,691,365, the entire contents of which are incorporated by reference. Other known antagonists/blockers include lophotoxin, neosurugatoxin, and erysodine (J. Med. Chem. (1997) 40:4169-4194). The skilled person will be aware of such nAChR antagonists/blockers, for example those disclosed within Nicotinic ACh Receptors Scientific Review, Tocris Scientific Review Series (Wonnacott, Tocris Scientific Review Series, 2014, https://www.tocris.com/literature/scientific-reviews).

Further examples of nAChR antagonists/blockers that are selective for the α4β2 receptor include: A 85380 dihydrochloride (3-[(2S)-2-Azetidinylmethoxy]-pyridine dihydrochloride), ABT 089 (2-Methyl-3-[(2S)-pyrrolidinylmethoxy]pyridine), ABT 594 ((R)-5-(Azetidin-2-ylmethoxy)-2-chloropyridine), Dianicline ((5aS,8S,10aR)-5a,6,9,10-Tetrahydro-7H, 11H-8,10a-methanopyrido[2′,3′:5,6]pyrano[2,3-d]azepine), RJR 2403 ((E)-N-Methyl-4-(3-pyridinyl)-3-buten-1-amine), TC 2559 (4-(5-ethoxy-3-pyridinyl)-N-methyl-(3E)-3-buten-1-amine) and Varenicline (7,8,9,10-Tetrahydro-6,10-methano-6H-pyrazino[2,3-h][3]benzazepine tartrate), or a pharmaceutically acceptable salt or derivative thereof.

### Pharmaceutical Compositions and Routes of Administration

An effective amount of a nAChR antagonist/blocker may be administered by various modes of administration, including for example rectal, buccal, intranasal and transdermal routes, by intra-arterial injection, intravenously, intrathecally, intracranially, intraperitoneally, intracerebroventricularly, parenterally, intramuscularly, subcutaneously, orally, topically, nasally, as an inhalant, or via an impregnated or coated device such as a stent.

Suitably, the nAChR antagonist/blocker is administered orally, nasally, intraperitoneally, intracerebroventricularly, intracranially, intrathecally, subcutaneously, or intravenously.

Suitably, the nAChR antagonist/blocker is able to penetrate the blood brain barrier. A nAChR antagonist/blocker that can penetrate the blood brain barrier may be administered by a number of routes such as those described herein.

Suitably, the nAChR antagonist/blocker is administered directly to the CNS. It is envisaged that administration directly to the CNS will reduce the risk of peripheral side effects associated with the administration of the nAChR antagonist/blocker.

Suitably, the nAChR antagonist/blocker is administered via local infusion to the CNS or direct injection to the CNS. Administration to the CNS may be achieved via intracranial infusion, for example intrastriatal infusion, intraputamenal infusion, intracaudate infusion, intraventricular infusion, intraparenchymal infusion and intracortical infusion.

According to a further aspect of the invention there is provided a pharmaceutical composition comprising a nAChR antagonist/blocker as defined hereinbefore, or a pharmaceutically acceptable salt thereof, and one or more pharmaceutically acceptable excipients.

Suitably, the pharmaceutical composition further comprises one or more additional pharmaceutically active agents, as defined herein.

Suitably, the pharmaceutical composition described herein is for use in the treatment of a condition or disorder defined herein.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing). The compositions of the invention may be in a form suitable for administration directly to the CNS (for example as a sterile aqueous or oily solution for local infusion to the CNS or direct injection to the CNS).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a nAChR antagonist/blocker for use in therapy is an amount sufficient to treat or prevent a condition in which reduced levels of dopamine in the CNS are implicated, referred to herein, slow its progression and/or reduce the symptoms associated with the condition.

The amount of active ingredient that is combined with one or more excipients to produce a single dosage form will necessarily vary depending upon the individual treated and the particular route of administration. For Example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a nAChR antagonist/blocker will naturally vary according to the nature and severity of the conditions, the age and sex of the animal or patient and the route of administration, according to well-known principles of medicine.

In using a nAChR antagonist/blocker for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a nAChR antagonist/blocker.

### Combinations

The nicotinic acetylcholine receptor antagonist/blocker treatment defined hereinbefore may be applied as a sole therapy or may involve, in addition to the nicotinic acetylcholine receptor antagonist/blocker, further agents that are conventionally used for the treatment of the condition concerned. Thus, the nicotinic acetylcholine receptor antagonist/blocker or pharmaceutical composition may be administered in combination with one or more additional pharmaceutically active agents.

Such conjoint treatment may be achieved by way of the simultaneous, sequential or separate dosing of the individual components of the treatment. Such combination products employ the nicotinic acetylcholine receptor antagonist/blocker within its approved dosage range described hereinbefore and the other pharmaceutically-active agent within its approved dosage range.

In a further aspect of the invention there is provided a nicotinic acetylcholine receptor antagonist/blocker, or a pharmaceutically acceptable salt thereof, in combination with another pharmaceutical agent for use in the treatment of a condition or disorder in which reduced levels of dopamine in the CNS are implicated. The condition or disorder may be any of those defined herein.

Suitably, the additional pharmaceutically active agent is an agent which increases the level of dopamine in the CNS by a different mechanism of action to the nicotinic acetylcholine receptor antagonist/blocker. Examples of such agents dopamine precursors (e.g. levodopa, co-careldopa, cobeneldopa), dopamine agonists (e.g. pramipexole, ropinirole, apomorphine, rotigine), MAO-B inhibitors (e.g. rasagiline, selegiline, safinamide), COMT inhibitors (e.g. entacapone, opicapone), amantadine, anticholinergics (e.g. procyclidine, trihexyphenidyl) or Ritalin.

In a further aspect of the invention there is provided a nicotinic acetylcholine receptor antagonist/blocker, or a pharmaceutically acceptable salt thereof, for use in the treatment of Parkinson's Disease, in combination with a dopamine precursor (e.g. levodopa, co-careldopa, cobeneldopa), a dopamine agonist (e.g. pramipexole, ropinirole, apomorphine, rotigine), a MAO-B inhibitor (e.g. rasagiline, selegiline, safinamide), a COMT inhibitor (e.g. entacapone, opicapone), amantadine or an anticholinergic (e.g. procyclidine, trihexyphenidyl). Suitably, the nicotinic acetylcholine receptor antagonist/blocker, or a pharmaceutically acceptable salt thereof, is for use in the treatment of Parkinson's Disease, in combination with a dopamine precursor (e.g. levodopa, co-careldopa, cobeneldopa).

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a nicotinic acetylcholine receptor antagonist/blocker, or a pharmaceutically acceptable salt thereof, in combination with an agent which increases the level of dopamine in the CNS by a different mechanism of action to the nicotinic acetylcholine receptor antagonist/blocker, in association with a pharmaceutically acceptable diluent or carrier.

In another embodiment, the invention relates to a therapeutic combination comprising a nicotinic acetylcholine receptor antagonist/blocker and a further agent which increases the level of dopamine in the CNS by a different mechanism of action to the nicotinic acetylcholine receptor antagonist/blocker.

### BRIEF DESCRIPTION OF THE DRAWINGS

Embodiments of the invention are further described hereinafter with reference to the accompanying drawings, which are discussed below:

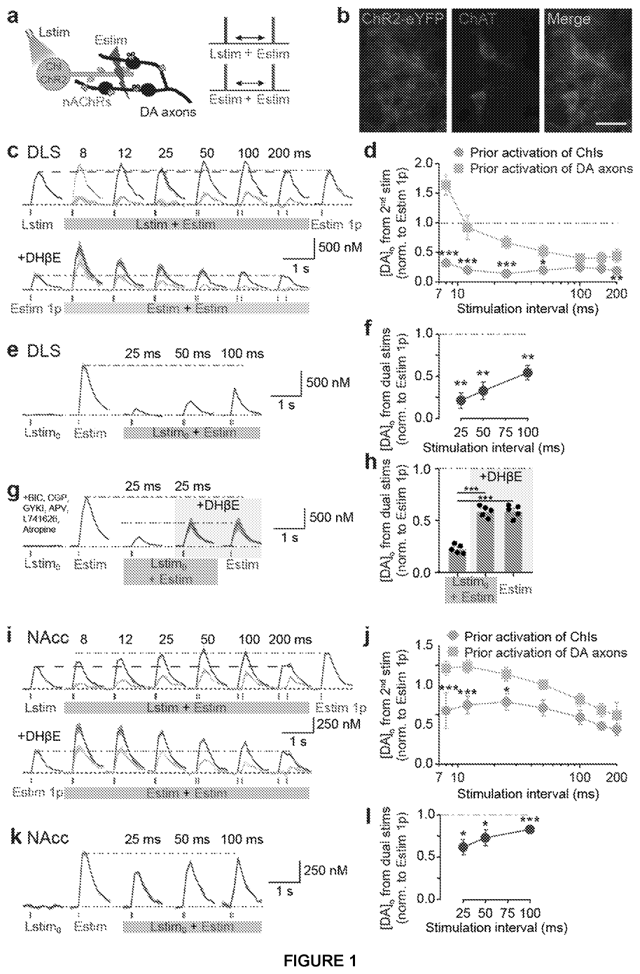

FIG. 1. Activation of ChIs inhibits subsequent DA release evoked by electrical stimulation.
a, Schematic of stimulation configuration for panels c-j. Blue light stimulation (Lstim) of ChR2-eYFP-expressing ChIs, local electrical stimulation (Estim) in striatal slices from ChAT-Cre: Ai32 mice.
b, ChR2-eYP expression in choline acetyltransferase (ChAT)-immunoreactive striatal neurons. Scale bar, 40 μm.
c,i, Upper, mean transients from representative experiments of [DA]ₒ (±SEM) evoked by a single pulse of Lstim (light grey lines, left side), or Estim (light grey lines, left side), or Estim (black lines, right side), or the composite response to paired Lstim plus Estim pulses (purple) at ISIs of 8-200 ms in DLS (c) and NAcc (i). Dark dashed trace, [DA]ₒ attributable to the paired Estim after subtraction of [DA]ₒ due to Lstim. Lower, mean transients of [DA]ₒ (±SEM) evoked by single or paired electrical pulses (dark grey lines) in the presence of DHβE (1 μM) in DLS (c) and NAcc (i). Light dashed trace, [DA]ₒ attributable to the paired Estim after subtraction of [DA]ₒ due to single Estim.
d,j, Mean peak [DA]ₒ (±SEM) evoked by the paired Estim normalised to [DA]ₒ evoked by a single Estim, versus ISI in DLS (d, N=5 animals) and NAcc (j, N=5 animals). *P<0.05, **P<0.01, ***P<0.001, Two-way ANOVA with Fisher's LSD test post hoc.
(e,k) Mean transients from representative experiments of [DA]ₒ (±SEM) evoked by a subthreshold light pulse (Lstim), single Estim, or paired Lstim and Estim at ISIs of 25-100 ms in DLS (e) or NAcc (k).
f,l, Mean peak [DA]ₒ (±SEM) evoked by the dual stimuli normalised to [DA]ₒ evoked by a single Estim, versus ISI in DLS (f, N=5 animals) and NAcc (l, N=5 animals). *P<0.05, **P<0.01, ***P<0.001, one sample t-test versus single electrical stimulation.

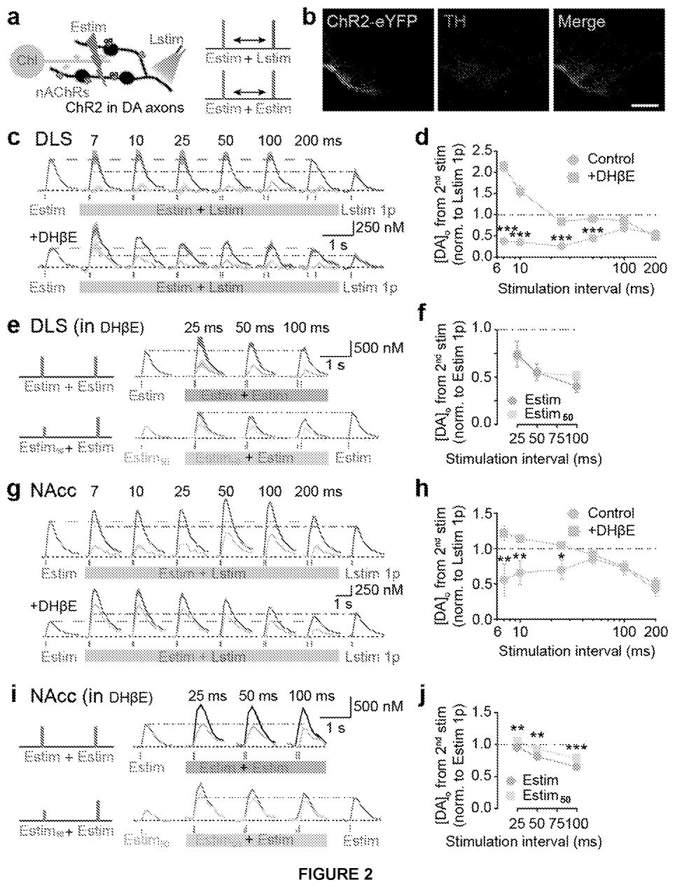

FIG. 2. Activation of ChIs inhibits subsequent DA release evoked by optogenetic stimulation
a, Schematic of stimulation configuration for panels m-t. Local electrical stimulation (Estim) in striatal slices and blue light stimulation (Lstim) of ChR2-eYFP-expressing DA axons.
b, ChR2-eYFP expression in midbrain DA neurons co-immunoreactive for DAT in DAT-Cre mice, after example VTA injection. Scale bar, 400 μm.
c,g, Mean transients from representative experiments of [DA]ₒ (±SEM) evoked by a single pulse of Estim (grey lines, left side) or Lstim (light grey lines, right side), or paired Estim plus Lstim pulses (dark grey) at ISI of 7-200 ms in DLS (c) and NAcc (g) when (upper) nAChRs can be active (no DHβE), or (lower) when nAChRs are inhibited (DHβE present) in DAT-Cre mice. Dark dashed trace, [DA]ₒ attributable to the paired Lstim after subtraction of [DA]ₒ due to Estim without DHβE, light dashed, [DA]ₒ attributable to the paired Lstim in the presence of DHβE.
d,h, Mean peak [DA]ₒ (±SEM) evoked by the paired Lstim normalised to [DA]ₒ evoked by a single Lstim, versus ISI in DLS (d, N=5 animals) and NAcc (h, N=5 animals). *P<0.05, **P<0.01, ***P<0.001, Two-way ANOVA with Fisher's LSD test post hoc.
e,i, Mean transients from representative experiments of [DA]ₒ (±SEM) evoked by a single full strength Estim (grey line, left side, upper), paired Estims (grey shading, upper), or a single low intensity Estim (Estim₅₀, light grey line, lower) with paired full-strength Estim (dark grey, lower) at ISIs of 25-100 ms in DLS (e) and NAcc (i) in wild-type animals. Light (lower) and dark (upper) dashed traces, [DA]ₒ attributable to the paired Estims after subtraction of [DA]ₒ due to Estim 1p.
f,j, Mean peak [DA]ₒ (±SEM) evoked by the paired stimulations versus ISI in DLS (F, N=5 animals) and NAcc (J, N=5 animals). DHβE was present in e, f, i, and j. **P<0.01, ***P<0.001, Two-way ANOVA with Fisher's LSD test post hoc.

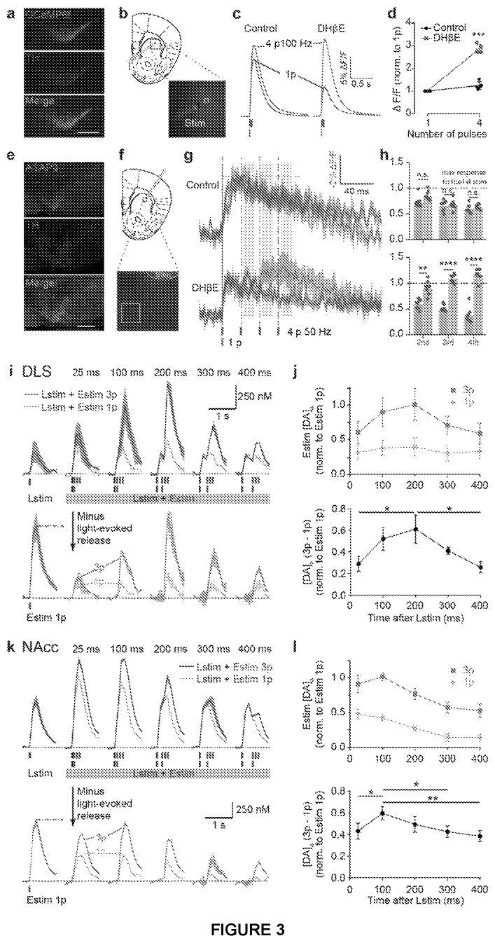

FIG. 3. Activation of ChIs and nAChRs inhibits repetitive axonal depolarisation and calcium accumulation in DA axons, and at longer intervals leads to apparent nAChR desensitisation
a, Images of VTA and SNc from DAT-Cre: Ai95D mice showing GCaMP6f-eGFP expression (top) in TH-immunopositive neurons (middle).
b, Illustration of stimulation configurations in live tissue.
c, Examples of Ca²⁺-imaging responses (changes to GCaMP6f fluorescence, ΔF/F) (mean±SEM from duplicates) in a DA axon population imaged in DLS in response to single or trains of 4 electrical pulses (4p) at 100 Hz in control conditions (left) and in the presence of DHβE (1 μM) (right).
d, Mean peak values (±SEM) for GCaMP6f ΔF/F vs. pulse numbers. Data are normalised to value for 1 pulse (N=5 animals). ***P<0.0001 paired t-test [DA]ₒ from 4 pulses of stimulation.
e, Images of VTA and SNc from DAT-Cre mice with ASAP3 expression (top) and TH-positive neurons (middle).
f, Illustration of stimulation configuration in live tissue.
g, Averaged transients of voltage sensor (changes to ASAP3 fluorescence, −ΔF/F, mean±SEM) in striatal dopamine axons to single or 4 electrical pulses at 50 Hz before and after blocking nAChRs with DHβE. Scale bar vertical axis applies to 1p data only. For the 4-pulse data, the peak value seen after the first pulse of stimulation is scaled to match the peak of transients seen for single pulses (n=7 recordings in N=4 animals).
h, The averaged responses at the time after the successive pulses in the pulse train (grey in G) before and after DHβE.
i,k, Mean transients from representative experiments of [DA]ₒ (±SEM) evoked by a single pulse of Lstim (left side, upper) or Estim (left side, lower), or Lstim paired with either 1 or 3 Estim pulses (labelled) at ISIs of 25-400 ms spanning activation, desensitisation, and resensitisation in DLS (i) and NAcc (k) in striatum of ChAT-Cre: Ai32 mice. Light and dark dashed traces, [DA]ₒ attributable to the paired stimuli after subtraction of Lstim.
j,l, Upper, mean peak [DA]ₒ (±SEM) evoked by paired Estims for 1p (circles) and 3p (squares) normalised to [DA]ₒ evoked by a single Estim, versus ISI, for DLS (j, N=5 animals) and NAcc (I, N=5 animals). Lower, differences between [DA]ₒ evoked by 3p and 1p Estims. *P<0.05, **P<0.01, One-way ANOVA with Tukey test post hoc.

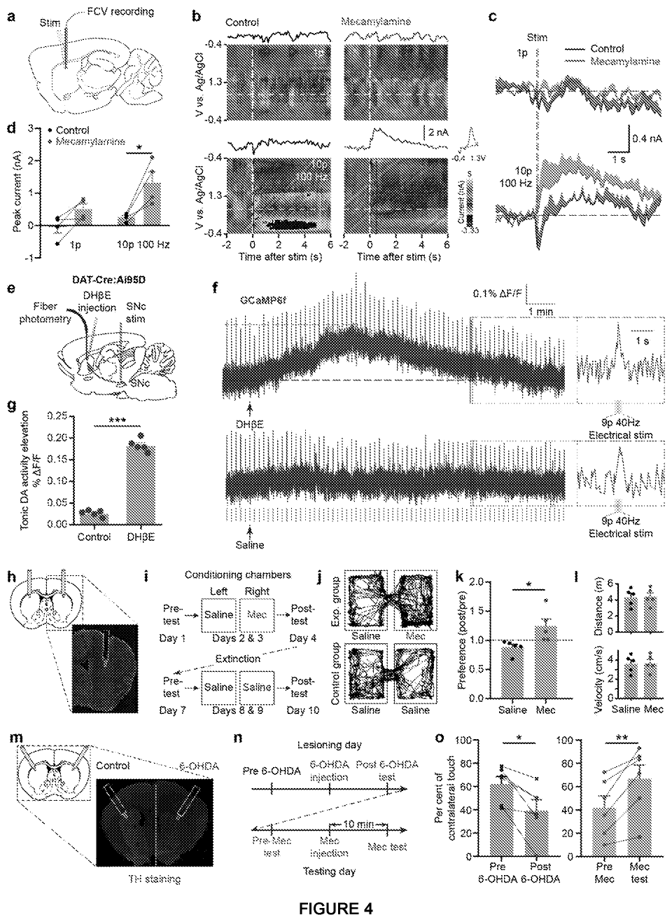

FIG. 4. nAChR antagonism in DLS in vivo promotes DA release, conditioned place preference and ameliorate motor deficits in mouse model of PD.
a, Schematic of configuration of recording and stimulation electrodes in DLS.
b, Colour plots and representative line plots of oxidation current (from dashed row at approximately +0.7 V) of voltammetric DA current vs. time and a corresponding DA cyclic voltammogram (from timepoint of black dotted line) before and after mecamylamine (i.p.).
c, DA signals (mean±SEM) evoked by 1 pulse or 10 pulses at 100 Hz before (black) and after (red) mecamylamine (2 mg/kg i.p.).
d, Mean peak DA currents (±SEM). **P<0.01 for Sidak's post-hoc multiple comparisons test (N=4 animals).
h, Schematic illustration of a bilateral cannula system for local infusion to dorsal striatum, and an example hemi-slice with DAPI staining.
i, Conditioning paradigm. Mice were conditioned for 4 times over two days (20 min per session) after local infusion of mecamylamine (10 μg/side, right chamber, grey) or saline (0.5 μl over 1 min, left chamber, black), whereas the control groups received saline for both chambers.
j, Representative tracking traces from the post-conditioning day.
k, Preference for the right chamber after conditioning. *P<0.05 paired t-test, N=5 animals in each test.
l, Total travel distances and velocity of movements in open-field test (N=5 animals).
m, Schematic illustration of TH-staining after unilateral 6-OHDA lesion.
n, Cylinder test paradigm. Mice were tested before and after 6-OHDA injection on lesioning day, and before and after mecamylamine infusion from the same cannula on testing day.
o, Striatal mecamylamine infusion reduces deficits in contralateral forelimb touching seen after lesion, *P<0.05, **P<0.01 paired t-test, N=6 animals in each test.

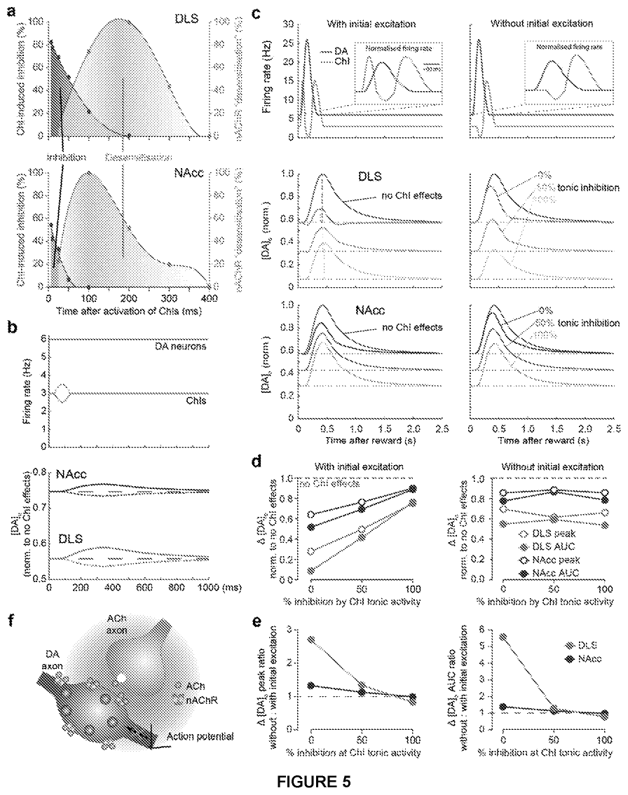

FIG. 5. ChI-induced inhibition dominates striatal DA release in a computational model.
a, Values used in the model for the strength of ChI-induced inhibition of DA release (dark gret) and the normalised level of apparent nAChR desensitisation (light grey) versus time after ChI activation in DLS (upper) and NAcc (lower).
b, When firing rate of DA neurons (labelled) is constant, a brief decrease (solid) or increase (dotted) of ChI activity (labelled) can respectively increase or decrease [DA]ₒ in DLS (labelled) and NAcc (labelled).
c, Top row, multiphasic ChI responses (labelled, light grey) with (left) or without (right) initial excitation phases, plus DA neuron burst activity (labelled, black) from¹⁶ were inputted to predict striatal DA release (lower rows) in DLS and NAcc when the tonic level of ChI-induced inhibition of DA release was set to 0% (labelled, upper middle line), 50% (labelled, lower middle line), and 100% (labelled, bottom line). [DA]ₒ are normalised to those seen with zero tonic ChI inhibition (labelled, no ChI effects).
d, Summary of peak [DA]ₒ (open circle) and area under [DA]ₒ curve (filled circle) in DLS (grey) and NAcc (black) with (left) and without (right) initial excitation in ChI multiphasic activity, when ChI-induced inhibition was set to 0%, 50%, and 100% at tonic activity of ChIs.
e, The ratio of peak [DA]ₒ (left) and area under [DA]ₒ curve (right) release when ChIs without and with initial excitation.
f, Schematic showing that activation of ChIs and nAChRs on DA axons limits action potential propagation.

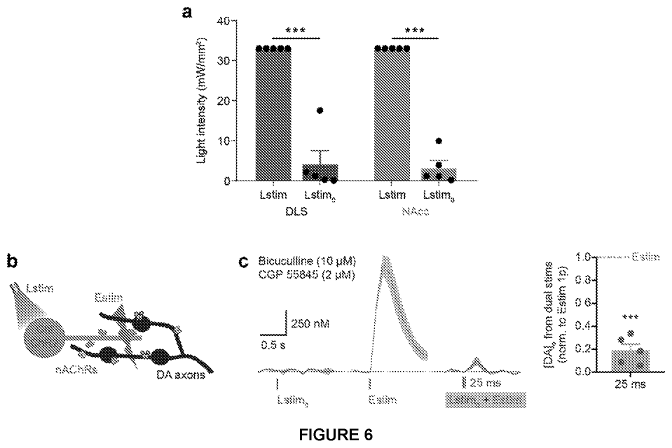

FIG. 6. Light stimulation (Lstim₀) intensities that are sub-threshold for evoking detectable DA release.
a, The intensities of Lstim₀ used to stimulate ChIs in ChAT-Cre: Ai32 mice in FIG. 1 were significantly lower than Lstim in both DLS (left hand side, dark grey) and NAcc (right hand side, grey). N=5 animals. ***P<0.001, one sample t-test.
b, Schematic of stimulation configuration for data in c. Blue light stimulation (Lstim) of ChR2-eYFP-expressing ChIs, subsequent local electrical stimulation (Estim) in striatal slices from ChAT-Cre: Ai32 mice.
c, Mean transients (±SEM) from representative experiments in DLS showing [DA]ₒ evoked by either a subthreshold light pulse (Lstim₀), a single Estim, or paired Lstim and Estim at an ISI of 25 ms when GABA_{A} and GABA_{B} receptors were inhibited using bicuculline (10 M) and CGP 55845 hydrochloride (2 μM) respectively. Mean peak [DA]ₒ (±SEM) evoked by the dual stimuli normalised to [DA]ₒ evoked by a single Estim (n=5 recordings, N=3 animals) when GABA receptors are inhibited. ***P<0.001, One sample t-test versus Estim.

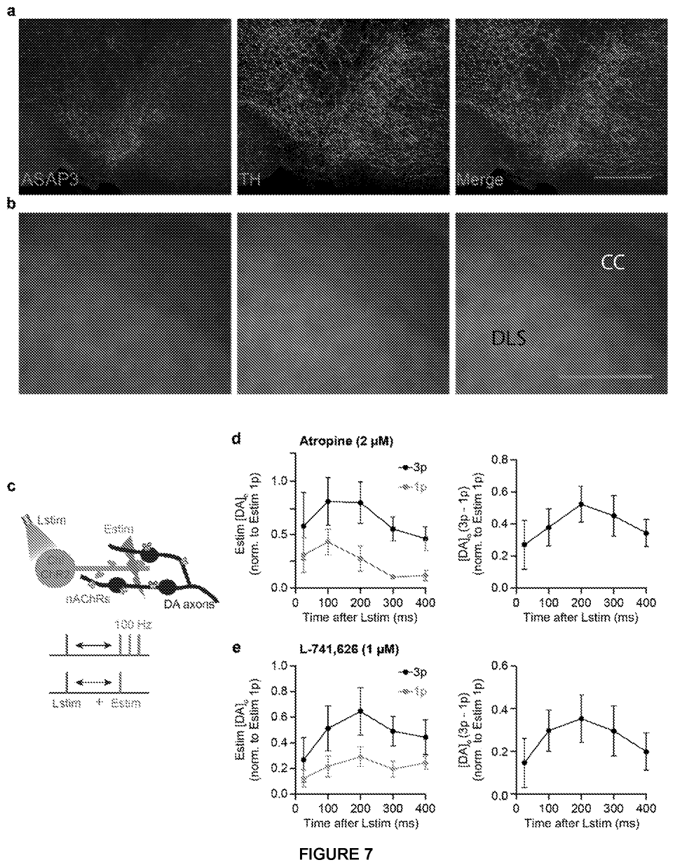

FIG. 7. Expression of ASAP3 in DA neurons and the dynamic DA release response after light activation of ChIs (100-400 ms interval) persists in DLS in the presence of antagonists for either muscarinic receptors or D₂ receptors.
a, Expression of ASAP3-GFP after viral injection in DA neurons within midbrain co-labelled for TH-immunoreactivity. b, the co-expression of ASAP3 and TH in dopamine axons in the DLS. CC: corpus callosum (scale bar, 400 μm). c, Schematic of stimulation configuration used in d and e. Blue light stimulation (Lstim) of ChR2-eYFP-expressing Chis, subsequent local electrical stimulation (Estim) in striatal slices from ChAT-Cre: Ai32 mice. d,e, Left, mean peak [DA]ₒ (±SEM) evoked in DLS after a Lstim by a subsequent Estim of either 1p (grey) or 3p 100 Hz (black) at a range of ISI, normalised to [DA]ₒ evoked by a single Estim, after antagonising either muscarinic receptors with atropine (2 μM) (d, n=5 recordings, N=3 animals) or D₂ receptors with L-741,626 (1 μM) (e, n=5 recordings, N=4 animals). Right, differences between [DA]ₒ evoked by 3p and 1p Estims. Significant effect of IPI seen in bell-shaped curves for both, P<0.001, One-way ANOVA.

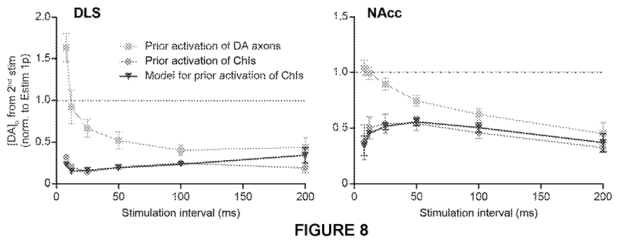

FIG. 8. The computational model predicts empirical ex vivo data.
The computational model built from empirical data in FIG. 1n,r predicted the impact of prior activation of Chis on subsequent DA release by an electrical stimulation as in FIG. 1d,h. Because the electrical stimulation (the second stimulation in FIG. 1d,h) evoked DA_{DA} and DA_{ChI} rather than only the DADA evoked by light stimulation (the second stimulation in FIG. 1n,r), the predicted data (triangle plot) was scaled as a whole curve to match the overall mean of the experimental data (circle plot) in DLS (left) and NAcc (right) to overcome the unknown ChI-induced inhibition of DA_{ChI} in this experimental paradigm.

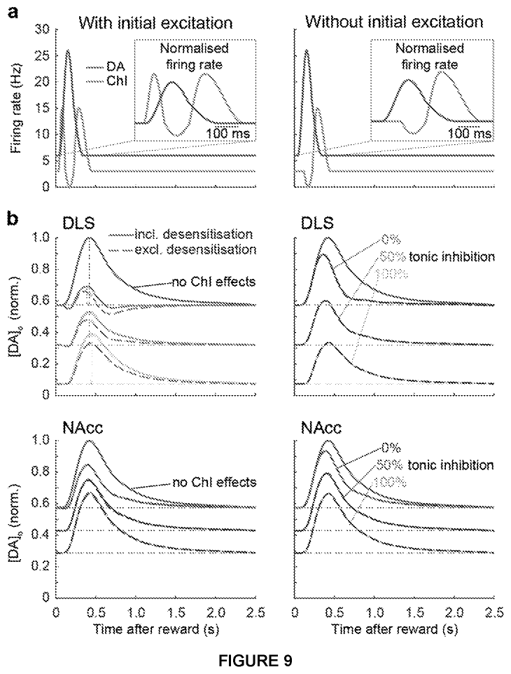

FIG. 9. “Desensitisation” of nAChRs plays a limited role in modifying phasic DA release in a computational model during multiphasic ChI activity.
a, Coincident phasic DA neuron firing (labelled, dark grey) and multiphasic ChI activity (labelled, light grey) with (left) or without (right) initial excitation in ChIs. Inset, firing rates normalised to baseline rates.
b, Phasic DA release was modified by ChI activity (compared to no ChI effects, labelled, top line) both with (solid line) and without (dashed line) a “desensitisation” component in the model (obtained from FIG. 2b) for DLS (upper) and NAcc (lower). Background levels of tonic inhibition of DA output by Chis included in the model were 0, 50 and 100%. Inclusion of a dynamic desensitisation component in the model impacted on phasic DA release only when there was initial ChI excitation (left), and only in DLS, acting to slightly increase phasic DA release by preventing nAChRs from inhibiting DA release during a rebound activity phase in ChIs. The effect is prominent in DLS (upper) than in NAcc (lower), consistent with the stronger ChI-induced inhibition of DA release in DLS than NAcc. As expected, excluding a dynamic desensitisation component from the model did not affect DA release when the initial excitation was absent because there was no initial increase in nAChR activation.

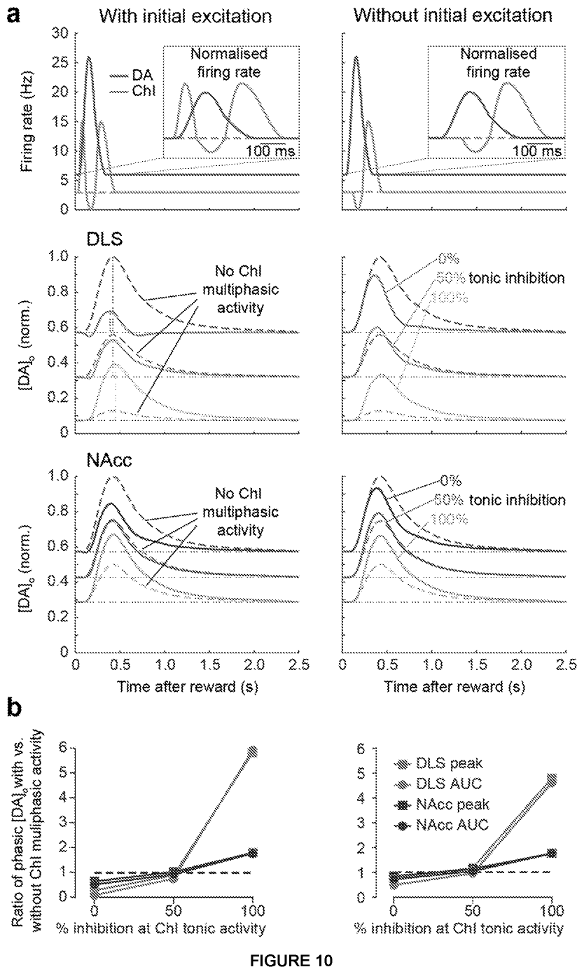

FIG. 10. The tonic level of ChI-induced inhibition of DA release modifies the relative impact of ChI multiphasic activity on phasic DA release in a computational model.
a, Upper, Model for coincident phasic DA neuron firing ((labelled, dark grey) and multiphasic ChI activity ((labelled, light grey) with (left) or without (right) initial excitation in ChIs. Lower panels, comparison of phasic DA signals at three different background levels of tonic inhibition of DA output (0, 50, 100%) before (dashed lines) versus after (solid lines) the development of multiphasic activity. At low-intermediate levels of tonic ChI inhibition of DA release (≤50%), multiphasic ChI activity reduced phasic DA release, whereas at very high levels of tonic ChI inhibition of DA release (50-100%), ChI multiphasic activity enhanced phasic DA release.
b, Summary of impact of multiphasic ChI activity on phasic DA release on different background levels of tonic inhibition of DA release by tonic ChI activity. Change to phasic DA signal amplitude and area under curve (AUC) with (left) or without (right) a phase of initial ChI excitation.

### DETAILED DESCRIPTION

Mesostriatal dopamine (DA) neurons play important roles in action selection and behavioural learning¹⁻⁴. Striatal DA release has long been assumed to be mainly determined by the action potentials generated at DA cell bodies in midbrain, but increasing evidence indicates that the dynamics of striatal DA release can be locally governed in DA axons⁵⁻⁷ and, in behaving animals, can be uncoupled from DA neuron firing in midbrain^{8, 9}. Striatal ChIs acting at nAChRs on DA axons could play critical roles^{5-7, 10, 11}. ChIs in vivo have dynamic tonic activity during spontaneous behaviour^{12, 13}, and also form multiphasic excitation-pause-rebound activity during learning, the so-called ‘pause response’ lasting 200-400 ms^{14, 15}, that coincides with phasic activity in DA neurons^{16, 17}. Somewhat paradoxically, ChI excitation and pauses have both been suggested to enhance DA release^{5, 6, 18}. Activation of a small population of ChIs drives DA release via nAChR activation^{5, 6}. In addition, a loss or pause in striatal nAChR activity, due to antagonism, desensitisation by nicotine, or deletion of forebrain ACh, promotes the frequency dependence of DA release^{7, 11, 18, 19}.

The inventors have noticed however, that the highest levels of DA output are evoked not when nAChRs are activated but when they are inactivated^{6, 7}. This long-overlooked outcome may suggest that there is a critical and unexplored negative impact on DA of activating nAChRs. Here, by activating Chis and DA axons ex vivo, in vivo and in silico, the inventors reveal that excitation of ChIs, at levels above or below the threshold to drive DA release, acts at nAChRs to inhibit subsequent DA release with short latency by preventing action potential-dependent depolarisation and Ca²⁺ summation in DA axons. Relieving this ChI-induced inhibition of DA release can remediate motor deficits in a mouse model of PD. The data provide a new framework to understand how ACh controls DA release, which is dominated by dynamic inhibition of DA following nAChR activation.

The inventors have identified that striatal cholinergic interneurons exert a powerful inhibition on dopamine axons to limit dopamine release after activating nicotinic acetylcholine receptors (nAChRs) on dopamine axons. This inhibition is particularly strong in the dorsal striatum, equivalent to caudate/putamen (CPu) in human, the area affected the most by the loss of dopamine neurons in the substantia nigra pars compacta (SNpc).

The present inventors have shown that blocking nAChRs can enhance dopamine signals ex vivo and in vivo and can induce conditioned place preference in mice. The administration of a nAChR antagonist/blocker will relieve the inhibition of dopamine release caused by striatal cholinergic interneurons. Compared to the traditional treatment of disorders such as Parkinson's, blocking of the nicotinic acetylcholine receptor will amplify physiological patterns of endogenous dopamine release that follow the firing pattern of dopamine neurons. The administration of nAChR antagonist/blockers is not expected to distort dopamine release like the dopamine precursor, levodopa, which likely provides a general elevation of dopamine level in the striatum, including through release from inappropriate non-dopaminergic inputs, and can lead to the development of profound side-effects after a few years. This new treatment will potentially prolong the treatment window of levodopa and reduce motor deficits in Parkinson's disease.

In the present invention, in mouse striatum it is shown that brief activation of ChIs and nAChRs subsequently and profoundly inhibits dopamine release with short latency (<7 ms) for 50-100 ms, and more profoundly in dorsolateral than ventral striatum. Inhibition of dopamine output is not due to depletion, but is reflected by intracellular calcium dynamics in dopamine axons. Correspondingly, antagonism of nAChRs in vivo promotes dopamine release in dorsal striatum and induces conditioned place preference. A computational model indicates that ChI-induced inhibition of dopamine release prevails during both multiphasic burst-pause and tonic activity in ChIs, particularly in dorsal striatum. The present invention therefore reveals that ChIs exert a region-specific influence on dopamine output that, contrary to the prevailing view, is predominantly inhibitory, with the power to uncouple dopamine release from somatic activity and critically shape dopamine function.

### Materials and Methods

### Animals

Male adult mice used in ex vivo experiments and in vivo DA recordings were C57BI6/J mice (Charles River, UK) (21-40 days), ChAT-cre:Ai32 (6-16 weeks), DAT-IRES-Cre (8-16 weeks), or DAT-Cre: Ai95D (4-7 weeks). ChAT-Cre^{+/+} mice (B6; 129S6-Chat^{tm2(cre)Lowl}/J, JAX stock number 006410) were crossed with Ai32^{+/+} mice (B6; 129S-Gt (ROSA) 26Sor^{tm32(CAG-COP4*H134R/EYFP)Hze/}J, JAX stock number 012569) to produce heterozygote ChAT-Cre: Ai32 mice. DAT-IRES-Cre^{+/-} mice (B6.SJL-Slc6^{a3tm1.1(cre)Bkmn}/J, JAX stock number 006660) were injected in midbrain with pAAV-double floxed-hChR2 (H134R)-EYFP-WPRE-pA for ChR2 expression in DA axons (8-16 weeks). DAT-IRES-Cre^{+/-} mice were crossed with Ai95D^{+/+} mice (B6:129S-Gt (ROSA) 26Sor^{tm95.1(CAG-GCaMP6)Hze}/J) to create heterozygote DAT-Cre: Ai95D mice.

Male C57BL/6N mice (Charles River, Beijing, China) (42-50 days) were used for behavioural experiments. Animals were group-housed and maintained on a 12-hour light/dark cycle with ad libitum access to food and water. The procedures for ex vivo recordings and anaesthetised in vivo DA recordings were performed in accordance with Animals (Scientific Procedures) Act 1986 (Amended 2012) with ethical approval from the University of Oxford, and under authority of a Project License granted by the UK Home Office. Behavioural experiments were performed using protocols approved by the Animal Care & Use Committees at the Chinese Institute for Brain Research (#CIBR-IACUC-007) and were performed in accordance with the guidelines established by US National Institutes of Health.

### Virus Injection

Heterozygote DAT-IRES-Cre mice were injected intracerebrally with a Cre-inducible recombinant AAV serotype 5 vector containing an inverted gene for channelrhodopsin-2 fused in-frame with a gene encoding enhanced yellow fluorescent protein (pAAV-double floxed-hChR2 (H134R)-EYFP-WPRE-pA) (titre=1E+12 vg/ml, University of North Carolina Vector Core), or a Cre-inducible recombinant AAV serotype 5 vector containing a EF1A promoter and an inverted gene for ASAP3 (titre=2.4E+12 vg/ml) without a soma-targeting signal and a WPRE RNA-stabilizing element (Stanford Gene Vector and Virus Core). Mice were placed in a stereotaxic frame under isoflurane anaesthesia and a craniotomy was made above the injection site. Injections of 1 L virus were given either unilaterally or bilaterally in either VTA (co-ordinates from Bregma in mm: AP−3.1, ML±0.5, DV−4.4) or in the SNc (from Bregma in mm: AP−3.5, ML+1.2, DV−4.0) using a 2.5 μL 33-gauge Hamilton syringe at 0.2 μL/min with a microinjector. The syringe was left in place for 5 min following each injection, then retracted slowly. Animals were maintained for at least 3 weeks following surgery to allow virus expression in striatum.

### Ex Vivo Slice Voltammetry

For fast-scan cyclic voltammetry (FCV) in acute coronal slices, animals were anaesthetised with isoflurane. Brains were quickly removed into ice-cold, high Mg²⁺ cutting solution containing in mM: 85 NaCl, 25 NaHCO₃, 2.5 KCl, 1.25 NaH₂PO4, 0.5 CaCl2, 7 MgCl₂, 10 glucose, 65 sucrose. Brains were then blocked, and 300 μm coronal slices were cut on a vibratome (Leica VT1200S) between +1.5 to +0.5 mm from bregma containing caudate-putamen and nucleus accumbens. Slices recovered at 32° C. for 30-40 min after dissection and were subsequently kept at room temperature. Slices were maintained and recorded in artificial cerebrospinal fluid (aCSF) containing in mM: 130 NaCl, 25 NaHCO₃, 2.5 KCl, 1.25 NaH₂PO₄, 2.5 CaCl₂), 2 MgCl₂, 10 glucose. The aCSF was saturated with 95% O₂/5% CO₂; recordings were made at 32-33° C. Extracellular DA concentration ([DA]ₒ) was measured using FCV with 7 μm-diameter carbon fiber microelectrodes (CFMs; tip length 50-100 μm) and a Millar voltammeter (Julian Millar, Barts and the London School of Medicine and Dentistry) as previously⁵⁵. The voltage was applied as a triangular waveform (−0.7 to +1.3 V range versus Ag/AgCl) at a scan rate of 800 V/s, and data were sampled at 8 Hz.

For optogenetic stimulations, ChR2-expressing Chis or DA axons were activated using a 473 nm diode laser (DL-473, Rapp Optoelectronic) coupled to the microscope with a fiber optic cable (200 μm multimode, NA 0.22). Spot illumination had a 30 μm diameter under ×40 immersion objective. Laser pulses were 2 ms duration, 23 mW/mm² at specimen. The Lstim₀ was achieved by lowering the laser intensity to the point at which there was no detectable evoked FCV signal above noise, in on-line or off-line analyses (Extended FIG. 1).

For electrical stimulations, 0.65 mA current (200 us width) was delivered through a surface bipolar concentric Pt/Ir electrode (125 μm outer, 25 μm inner diameter, FHC, USA) placed ˜100 μm from the recording electrode. The Estim₅₀ was the stimulation current at which evoked [DA]ₒ was ˜50% of that seen with normal stimulation (0.65 mA) during on-line analysis. Stimulations were timed to avoid FCV scans.

### Calcium Imaging

As in a previous study²¹, an Olympus BX51WI microscope equipped with a OptoLED Lite system (CAIRN Research), Prime Scientific CMOS (sCMOS) Camera (Teledyne Photometrics), and a ×40/0.8 NA water-objective (Olympus UK) was used for wide-field fluorescence imaging of GCaMP6f in dopaminergic axons in DLS in ex vivo slices in response to single and trains (4 pulses, 100 Hz) of electrical stimulus pulses. Images were acquired at 16.6 Hz frame rate every 2.5 min using Micro-Manager 1.4, with stimulation and recording synchronised using custom-written procedures in Igor Pro 6 (WaveMetrics) and an ITC-18 A/D board (Instrutech). Image files were analysed with Matlab R2019b and Fiji 1.5. The inventors extracted fluorescence intensity from the region of interest (ROI) and from an equal background area where there was no GCaMP6f expression (on the stimulating electrode). After background subtraction, the Ca²⁺ transients were bleach-corrected by fitting an exponential curve function through both the baseline (2 s prior to stimulation) and the last 1 s in a 7.2 s recording window. The order of single and train stimulations was alternated and equally distributed and data were collected in duplicate before and after a change in extracellular experimental condition. Data are expressed as ΔF/F where F is the fitted curve.

### Voltage Sensor Imaging

An Olympus BX51WI microscope equipped with a OptoLED Lite system (CAIRN Research), an iXon EMCCD Camera (ANDOR), and a ×40/0.8 NA water-objective (Olympus UK) was used for wide-field fluorescence imaging of ASAP3 in dopaminergic axons in DLS in ex vivo slices in response to single and trains (4 pulses, 500 Hz) of electrical stimulus pulses. Images were acquired at 660 Hz frame rate every 2.5 min using Micro-Manager 1.4, with stimulation and recording synchronised using PClamp. Image files were analysed with Matlab R2019b and Fiji 1.5. The inventors extracted fluorescence intensity from the region of interest (ROI). The ASPA3 transients were bleach-corrected by fitting an exponential curve function. The order of single and train stimulations was alternated and equally distributed and data were collected in duplicate before and after a change in extracellular experimental condition. Data are expressed as ΔF/F where F is the fitted curve.

### Anesthetised In Vivo Voltammetry

Wild type mice were anesthetised with urethane (1.4-1.9 g/kg, i.p.; Biolab), supplemented with additional urethane (0.2 g/kg) every 1-2 hr as required. All wounds and pressure points were infiltrated with bupivacaine (0.5%). Upon reaching surgical anaesthesia, the head was fixed in a stereotaxic frame (Kopf, USA). Core temperature was maintained at 35-36° C. using a homeothermic blanket and monitored via a rectal probe (TR-100, Fine Science Tools). A round piece of skull overlying the left hemisphere was removed to target the DLS (AP+1.0 mm, ML 1.6 mm, DV 2.2 mm to Bregma). A stimulating and recording array consisting of a carbon-fiber microelectrode and a bipolar stimulating electrode (MS303/3-A/SPC, P1 Technology) was positioned in the DLS. The Ag/AgCl reference electrode was implanted in another part of the forebrain. [DA]ₒ was measured using FCV with 7 μm-diameter carbon fiber microelectrodes (CFMs; tip length 50-100 μm) and a Tarheel system (University of Washington, Seattle, US). The voltage was applied as a triangular waveform (−0.4 to +1.3 V range versus Ag/AgCl) at a scan rate of 400 V/s and data were sampled at 10 Hz. The location of the tip of FCV electrode was confirmed histologically.

For striatal electrical stimulation, 0.65 mA current (200 μs) was delivered through a bipolar stimulating electrode (0.005 inch, MS303/3-A/SPC, P1 Technologies). The stimulating electrode tips were separated by ˜500 μm and were glued to the FCV recording electrode to fix the tip of the FCV electrode between the two stimulating poles.

### Behavioural Recordings

Cannulae Placements: Male C57BL/6N mice (42-50 days) were anesthetised with isoflurane (5% induction; 1.5-2% maintenance) and placed on a stereotaxic frame for surgery. Bilateral injection needles (O.D. 0.21 mm, I.D. 0.11 mm, RWD, China) with the guide cannula (O.D. 0.41 mm, I.D. 0.25 mm, RWD, China) were implanted to the dorsal striatum either vertically or at a small angle from the vertical, with the tip of each cannula aimed at coordinates: AP+1.0 mm; ML+/−1.6 mm to bregma; DV-2.4 mm (from dura). Mice recovered for three days after surgery.

Conditioned Place Preference Testing: In CPP experiments, mice were placed in a 40 cm×40 cm transparent plexiglass arena which was divided into two equal chambers separated by doorway. The chambers were decorated with either horizontal or vertical stripes. The movement of animals was recorded and analysed with Smart V3.0 tracking software (Panlab, Spain).

On day 1, mice were allowed to freely shuttle between two chambers to assess place preference at baseline, expressed as % time spent in right chamber. The mice were conditioned on days 2 and 3, when animals received alternating bilateral striatal injection with either mecamylamine (10 μg/side) or saline vehicle (0.9%) in a volume of 0.5 μl over 1 min in AM and PM. Animals were then constrained respectively in the right or left chamber for 20 min. The treatments were counterbalanced for time of day. On day 4, the post-conditioning chamber preference was calculated as the % of time spent in the right mecamylamine-associated chamber compared to on pre-conditioning day 1. For the next two days (days 5-6), animals received bilateral saline injection and explored both chambers for 20 min after which place preference was extinguished. The conditioning procedure was then repeated but for bilateral saline for both chambers, with a pre-conditioning test on day 7, two days of conditioning on days 8-9, and a post-conditioning test on day 10. To minimise place preference bias at baseline, the five animals in each test showing least place preference on the pre-conditioning day (mecamylamine 42%-58%; control 45%-55%) were selected for subsequent conditioning. For open field experiments, mice received bilateral striatal injection of either saline vehicle or mecamylamine (10 μg/side), and were placed into the open field chamber to assess total running distance and average velocity within 20 minutes.

6-OHDA Lesioning and Reaching Task: On the lesioning day, 6-hydroxydopamine was injected into the right dorsolateral striatum through the pre-implanted cannula (1 μl of a 0.9% saline solution containing 4 μg/μL 6-OHDA (Sigma-Aldrich) and 0.02% acetic acid) over 5 min. On the testing day, mecamylamine (10 μg/side) was injected via the cannula in a volume of 0.5 μl over 1 min into the lesioned (right) striatum. Animals were sacrificed immediately after behavioural testing. The 6-OHDA-induced loss of dopamine axons was confirmed by the ˜50% decrease of fluorescence intensity of TH staining in slices compared to the contralateral DLS which was measured with ImageJ (1.52p). The anterior commissure used as 0% background fluorescence. Animals were briefly sedated with isoflurane during the injection of 6-OHDA and mecamylamine.

A cylinder test was performed before and 10 min after 6-OHDA injection to confirm an acute effect of 6-OHDA. After one day of lesion 56, the test was performed again before and 10 min after mecamylamine injection. No habituation of the mice to the cylinder was conducted. During the test, mice were individually placed into a glass cylinder (diameter 19 cm, height 25 cm). Mirrors were placed behind the cylinder to fully view and record wall touches. Only wall touches with a single paw, ipsilateral or contralateral forelimb, were counted in the analysis. Data are contralateral touches expressed as a percentage of total number of single-paw touches.

### Immunocytochemistry

After voltammetry recordings in acute slices, slices were fixed in 4% paraformaldehyde dissolved in PBS containing 0.2% picric acid. Slices were fixed overnight at 4° C. and then stored in PBS. Free-floating sections were then washed ×5 in PBS for 5 min and incubated in 0.5% Triton X-100 and 10% normal donkey serum.

Chis expressing ChR2-eYFP were identified as ChAT-immunoreactive as previously (Zhang et al., 2018). Fixed and rinsed slices were incubated overnight with goat anti-ChAT 1:100 antibody (Millipore) dissolved in PBS containing 0.5% Triton X-100 and 3% normal donkey serum. Sections were then washed ×5 with PBS for 5 min and incubated for 2 hr at room temperature with 1:1000 Alexa Fluor 568 donkey anti-goat antibody (Invitrogen) dissolved in PBS containing 0.5% Triton X-100 and 3% normal donkey serum.

DA neurons co-expressing ChR2-eYFP, GCaMP6f-eYFP or ASAP3 and striatal DA axons were identified by immunoreactivity to tyrosine hydroxylase (TH) as previously 21. Fixed and rinsed slices were incubated overnight with 1:2000 rabbit anti-TH antibody (Sigma) dissolved in PBS containing 0.5% Triton X-100, 1% normal goat serum and 1% foetal bovine serum. Sections were then washed ×5 with PBS for 5 min and incubated for 2 h at room temperature with 1:1000 (DyLight 594 goat anti-rabbit antibody (Jackson) dissolved in PBS containing 0.5% Triton X-100, 1% normal goat serum and 1% foetal bovine serum.

Sections processed as above were then washed with PBS and mounted on gelled slides with Vectashield mounting medium (Vector Labs) and imaged at 20×, N.A. 0.8, using a Zeiss LSM880 confocal microscope system running ZEN black version 2.3 (Zeiss), or on a confocal microscope system (FV1000 IX81; Olympus) using a 20×/0.75 NA objective and Fluoview software (Olympus). Maximum intensity projection from a z-stack of height 30 μm was captured individually and the stack of the pictures were compressed. Red fluorescence (TH and ChAT) was captured at 638-759 nm with 633 nm excitation. Green fluorescence (GCaMP, ChR2, and ASAP3) was captured at 493-630 nm with 488 nm excitation.

To verify carbon-fibre locations in dorsal striatum for in vivo FCV recordings, anaesthetised mice were sacrificed and brains were quickly removed and fixed in 4% paraformaldehyde (PFA) overnight. The fixed brains were then sectioned into 50 μm slices using a Vibratome (Leica). Slices were rinsed with PBS 3 times and mounted on glass slides and then imaged under microscope to identify the location of recording sites.

To verify placements of intrastriatal injection cannulae and 6-OHDA lesion in behavioural experiments, mice were anesthetised with i.p. injection of Avertin (250 mg/kg body weight) and transcardially perfused with saline and 4% PFA. Brains were dissected and post-fixed overnight in 4% PFA then dehydrated by 30% sucrose for 24 hours. The fixed brains were then frozen-sectioned into 50 μm slices using a Vibratome (Leica). Slices to verify the placement of cannulae were rinsed ×3 with PBST (PBS containing 0.1% Tween 20) and mounted on glass slides with 5 μg/ml DAPI. Slices to verify 6-OHDA were stained with immunoreactivity to TH as slices from ex vivo experiments. The slices were imaged under inverted confocal microscope (Zeiss) with 405 nm laser for excitation.

### Drugs

DHβE and mecamylamine hydrochloride for ex vivo and anaesthetised in vivo experiments were purchased from Tocris Bioscience (UK). Mecamylamine hydrochloride for behavioural experiments were purchased from Sigma Aldrich (China). All other chemicals were purchased from Sigma Aldrich (UK). Pharmacological drugs for ex vivo experiments were prepared in distilled deionised water as stock aliquots at 1000× final concentrations and stored at −20° C. Drug stocks were then diluted to the final concentration in carbogenated aCSF immediately before use and were bath-applied. Drugs for in vivo experiments were dissolved in sterilised saline to final concertation.

### Computational Model

The computational model was written in MATLAB. The model included: (1) the dynamic strength of ChI-induced inhibition, determined from the ratio of [DA]ₒ evoked at a second stimulus before and after inhibiting nAChRs with DHβE (from FIG. 1n,r; DAT-Cre, light stimulus) fitted to a polynomial curve; (2) the profile of apparent nAChR “desensitisation” (FIG. 3e,g) estimated from the change in the difference between [DA]ₒ evoked by 3 pulse and 1 pulse at a second stimulus (from FIG. 3f,h), normalised to a maximum and fitted with polynomial curves; and (3) the dynamic release and uptake profile of [DA]ₒ seen after a single electrical stimulation ex vivo in order to model [DA]ₒ in vivo as a scalar product with DA neuron activity. The inventors also included in the model a range of potential levels of background tonic ChI-induced inhibition of DA release (0, 50, 100%) arising from tonic activity in ChIs. The inventors excluded a potential component of DA release that can be driven by synchronised activation of ChIs in some experimental scenarios (FIG. 1)^{5, 6}, since it was found that the threshold for nAChR-mediated inhibition of DA release is lower than that required to drive release ex vivo (see FIG. 1) and was met in vivo after discrete striatal stimulation (see FIG. 4). The strength of ChI-induced inhibition (1) was a best-fit polynomial curve fitted to the ratio of [DA]ₒ before and after inhibiting nAChRs in FIG. 1n,r. (DLS: y=1.97e+5x²−0.00833x+0.872, R²=0.99; NAcc: y=8.71e+5x²−0.0149x+0.611, R²=0.96). The level of nAChR desensitisation (2) was obtained from FIG. 4 b,d and was normalised to its own maximum and minimum. The normalised data was then best-fitted with a polynomial curve (DLS: y=2e-05x²−0.0087x+0.88 NAcc: y=−9e-10x⁴+9e-07x³−0.0003x²+0.039x−0.5654).

### Quantification and Statistical Analysis

Statistical analyses used GraphPad Prism 6.0. Data are expressed as mean±standard error of the mean (SEM). The N value is the number of animals, n value is the number of individual recordings. One-sample t test, t-test, and two-way ANOVA were used.

### Results

### Activation of Chis Inhibits Subsequent DA Release

The inventors tested the impact of activation of Chis on subsequent DA release. In ex vivo striatal slices from ChAT-Cre: Ai32 mice (FIG. 1a,b), The inventors used targeted optogenetic activation of ChR2-expressing Chis to activate nAChRs and drive DA release (ChI-activated DA release, DA_{ChI}) 6 followed 8-200 ms later by an electrical stimulus pulse to drive DA release both directly (DADA) and indirectly via activation of ChIs (DA_{ChI}, which follows with short latency, ˜10 ms^{10, 20}). In both dorsolateral striatum (DLS) (FIG. 1c,d) and nucleus accumbens core (NAcc) (FIG. 1i,j), targeted activation of ChIs evoked DA release and then depressed subsequent DA release evoked electrically 8-200 ms later, to ˜15-50% of peak extracellular DA concentrations ([DA]ₒ) evoked by an electrical stimulus alone. For inter-stimulus intervals≤50 ms in DLS and ≤25 ms in NAcc, this depression was significantly greater than produced by an initial electrical stimulation in the presence of a nAChR antagonist (DHβE, 1 μM) (FIG. 1c,d,i,j), for which there was a strong inverse relationship between inter-stimulus interval and [DA]ₒ, as seen previously^{7, 21}. These data suggest that nAChRs inhibit subsequent DA release on short timescales. D₂ autoreceptors on DA axons can be excluded as a contributing mechanism because D₂ receptors only operate after intervals longer than 200 ms²¹. The Inventors then tested whether the apparent inhibition of subsequent DA release seen after ChI activation involved a suggested depletion of the DA vesicle pool¹⁰. The Inventors activated ChIs with a low light intensity (Lstim₀) (FIG. 6) that was below the minimum intensity required to trigger detectable DA release (<0.5% of DA_{ChI} evoked by normal Lstim) (FIG. 1e,k). Activation of Chis using Lstim₀, continued to depress [DA]ₒ evoked by a subsequent electrical pulse, to as little as 20-60% (DLS) or 60-80% (NAcc) of [DA]ₒ evoked by an electrical pulse alone (FIG. 1e, f, k,l). Therefore, activation of ChIs/nAChRs, at levels lower than those required to drive DA release, inhibits subsequent DA release independently of the DA vesicle pool or prior DA release level. Inhibition of DA release was not mediated by striatal GABA, which can arise from co-release from ChIs²² or DA axons²³, GABA neurons and from GABA tone²⁴, because it persisted in the presence of GABA receptor antagonists (FIG. 6).

The Inventors also explored whether prior ChI activation limits subsequent DA release when driven by targeted optogenetic activation of DA axons (DA_{DA}). The Inventors used an electrical stimulus to initially drive DA release (DA_{DA}+DA_{ChI}) and then assessed DADA driven by a subsequent light activation of ChR2-expressing DA axons in ex vivo striatal slices from DAT-Cre mice (FIG. 2a,b). In both DLS (FIG. 2c,d) and NAcc (FIG. 2g,h), an electrical stimulus evoked DA release but then significantly depressed [DA]ₒ evoked by a subsequent light pulse compared to [DA]ₒ evoked by a single light pulse alone. For inter-stimulus intervals <100 ms (DLS) or <50 ms (NAcc), this depression was relieved when a nAChR antagonist was present (DHβE, 1 μM). The relief from depression was not a consequence of lower levels of initial DA release (approximately half): in wild-type mice, the Inventors reduced initial DA release to a half by lowering electrical stimulation intensity (Estim₅₀) but found that the level of [DA]ₒ evoked by a subsequent full intensity electrical stimulation was the same as following a full strength stimulus in DLS (FIG. 2 e,f), and was only slightly greater in NAcc (FIG. 2 i,j).

Therefore, activation of nAChRs by Chis can inhibit DA release during subsequent activation of DA axons through a mechanism independent of DA pool availability, or subsequent ACh release. This mechanism persists for durations equivalent to ChI multiphasic pauses and is more pronounced and longer lasting in DLS than NAcc.

Activation of nAChRs Limits Axonal Ca²⁺ Summation and Subsequent Depolarisation

DA release is strongly governed by axonal activation mechanisms upstream of Ca²⁺ entry, as well as those governing Ca²⁺ entry, intracellular buffering and signalling^{21, 25, 26}. The Inventors tested whether striatal nAChRs limit axonal Ca²⁺ summation during successive pulses. In brain slices of DAT-Cre: Ai95D mice (FIG. 3a,b), the Inventors imaged Ca²⁺ reporter GCaMP6f in DA axons in DLS as previously²¹ evoked by single pulses versus 4-pulse trains (100 Hz), with and without nAChR antagonism. This stimulus protocol indicates nAChR activation: [DA]ₒ evoked by pulse trains is only slightly greater than for single pulses when nAChRs can be activated, but markedly greater when nAChRs are antagonised^{7, 27}. In parallel, axonal GCaMP6f fluorescence was only slightly greater for a train compared to a single pulse when nAChRs were active (FIG. 3c,d), but markedly greater when the Inventors antagonised nAChRs (DHβE, 1 μM) (FIG. 3c,d). These data indicate that nAChR activation limits summation of axonal [Ca²⁺]; in response to subsequent stimuli.

To test whether nAChRs directly limit axonal depolarisation during successive stimulation of dopamine axons, the Inventors imaged the ASAP3 voltage sensor²⁸ expressed in DA axons in DLS in brain slices during single pulses or 4-pulse (50 Hz) electrical stimuli (FIG. 3e,f, FIG. 8). In the absence of an nAChR antagonist, dopamine axons were depolarized by the first pulse but evidently not by subsequent pulses in a train (FIG. 3g,d). In contrast, when nAChRs were antagonised, DA axons were able to depolarise on each successive stimulus pulse during a train (FIG. 3g,h). These results indicate that activation of nAChRs limits subsequent dopamine release with short latency by preventing subsequent spike-induced depolarisation and Ca²⁺ entry in dopamine axons.

nAChRs Activated by Initial Excitation are Off During ChI Rebound

In a multiphasic response, Chis show a ‘rebound’ increase in activity some ˜100-300 ms after initial excitation^{16, 29}. At these intervals, the short latency ChI-induced inhibition of DA release has dissipated (see FIG. 1). However, for the ensuing ˜1 second, further DA release can only be driven by direct optogenetic activation of DA axons but not ChIs^{6, 10}. This apparent refractoriness of DA release driven by Chis but not DA axon activity suggests that nAChRs on DA axons are unresponsive to ACh released at the time of rebound ChI activity. Desensitisation of nAChRs³⁰⁻³² has been suspected to play a role in DA release dynamics during stimulus trains³³. The Inventors tested whether the DA release dynamics at the timepoints of ChI rebound activity are consistent with nAChR desensitisation. The Inventors exploited the observation that activation of nAChRs limits DA release at subsequent pulses (as in FIG. 1), whereas the turning off or desensitisation of nAChRs will promote subsequent release during a train⁷. As a potential readout of the timecourse of nAChR desensitisation, the Inventors explored how the difference in [DA]ₒ evoked by triplet versus single pulses (100 Hz) varied over time. In striatal slices from ChAT-Cre: Ai32 mice, the Inventors light-activated ChIs, then 25-400 ms later, applied either single or triplets of electrical pulses (100 Hz) (FIG. 3 i,j, also see FIG. 7c). The difference in [DA]ₒ evoked by triplet versus single pulses varied with interval, peaking at ˜200 ms in DLS and ˜100 ms in NAcc, and decaying by 400 ms (FIG. 3 i,j,k,l). Neither muscarinic nor D₂-receptor activation were responsible for these dynamics (FIG. 7d,e). These dynamics could reflect the timecourse of nAChR desensitisation and resensitisation, with regional differences reflecting different nAChR stoichiometries^{34, 35}, and indicating that nAChRs will be at least partly desensitised during ChI rebound activity ˜100-300 ms after initial excitation.

nAChR Antagonism In Vivo Promotes DA Release and Induces Conditioned Place-Preference

The Inventors explored the impact of striatal nAChRs in vivo, when the balance of nAChR-mediated excitation vs inhibition of DA, or desensitisation, might differ from ex vivo. In urethane-anesthetised wild-type mice, injection of nAChR antagonist in DLS significantly increased [DA]ₒ evoked by local striatal stimulation with brief electrical pulse trains (FIG. 4 a-d), indicating that nAChRs in vivo can be activated to inhibit DA release; their properties are not dominated by desensitisation. Further, the Inventors tested in freely moving mice whether inhibiting nAChRs by bilateral injection of mecamylamine in dorsal striatum could promote reward-related learning³⁶. After two days of daily conditioning, mice developed a conditioned-place preference for the chamber conditioned with intrastriatal mecamylamine but not saline controls (FIG. 4 h-l). The findings are consistent with previous in vivo studies in adjacent NAc showing that nAChR antagonists increase reward-evoked DA levels and promote reward-related learning^{37, 38}. These data indicate that nAChR-induced inhibition of DA function predominates over directly triggering axonal DA release and nAChR desensitisation.

nAChR Antagonism Reverses Motor Deficits in a PD Model

These data raise the prospect that antagonists of nAChRs, by preventing ChI-induced inhibition of DA release, might have potential to offset motor deficits in PD arising from DA deficits. The Inventors tested whether striatal antagonism of nAChRs could remediate motor deficits induced by a DA lesion in mice. Striatal DA inputs in wild-type mice were unilaterally partially lesioned with local injection of 6-OHDA in the right dorsolateral striatum which led to reduction of striatal tyrosine hydroxylase-immunoreactivity. The Inventors tested the effect of nAChR antagonist on single paw reaching movements³⁹ in the contralateral limb (FIG. 4 m-o). Striatal injection of mecamylamine remediated these motor deficits (FIG. 4 m-o), restoring reaching movements to normal levels, indicating that nAChR antagonists might be a potential treatment to offset the impact of DA deficits in PD.

### Tonic and Multiphasic Activity in Chis Inhibits DA Release in a Computational Model

To understand how nAChR dynamics impact on [DA]ₒ in vivo during dynamic activity patterns of ChIs, the inventors developed a computational model based on data from FIGS. 2c&d and 2g&h (FIG. 5a). The Inventors validated that the model could simulate ex vivo observations of FIG. 1d&h (FIG. 8). The Inventors firstly tested how changing tonic ChI firing rate would modulate tonic [DA]ₒ. The Inventors set an arbitrary tonic level of ChI-induced inhibition of DA release (50% of maximum), and normalised [DA]ₒ to the level seen without ChI effects. The model predicted that transient (˜100 ms) changes in ChI population activity will generate opposite changes in [DA]ₒ in the absence of underlying changes in DA firing activity (FIG. 5b). This prediction could explain the recent observations in vivo that [DA]ₒ can be modified without an underlying change to DA neuron firing rate⁸.

The Inventors then tested how multiphasic ChI activity modifies [DA]ₒ during concurrent burst activity in DA neurons¹⁶, both with and without the initial excitation that occurs in half of ChIs²⁹, incorporating a range of levels of tonic nAChR-induced inhibition of DA release (0, 50, 100%) prior to multiphasic activity. Tonic ChI activity reduced baseline DA release levels (FIG. 5c). Moreover, for DA neuron burst activity, DA release was lowered by multiphasic activity in ChIs, to the greatest extent when accompanied by initial ChI excitation and lower tonic ChI activity, and in DLS more than in NAc (FIG. 5 c-e). The reduction in burst DA release seen during multiphasic ChI activity without initial excitation results from inhibition of DA release by concurrent ChI rebound activity. A desensitisation-like component played only a small role in these outcomes, and mostly in DLS not NAc, and when initial excitation was present not absent (FIG. 9). Overall, the model suggests that multiphasic activity in ChIs inhibits DA release during phasic activity, particularly in DLS.

The model also indicated that tonic ChI activity in the absence of multiphasic ChI activity, reflective of scenarios prior to learning^{29, 40}, reduces baseline and burst-evoked levels of DA release (dashed lines, FIG. 10a). At low-intermediate levels of tonic ChI inhibition of DA release (<50%), multiphasic ChI activity further reduced phasic DA release. Only at extremely high levels of tonic inhibition (50-100%), did ChI multiphasic activity enhance phasic DA release (FIGS. 10a and b), presumably owing to lowering of ChI inhibition during a ChI pause. Therefore, the level of phasic striatal DA release will be a function of both tonic and multiphasic activity in ChIs.

### Discussion

The inventors reconcile the diverse actions of nAChRs on DA release and revise current thinking to show that nAChR activation leads to a dominant inhibition of DA release. The results described herein demonstrate that DA output is gated by the dynamics of nAChR activation via a rapid onset but longer duration inhibition of DA release (<100 ms), plus a subsequent apparent loss of nAChR control (after ˜100-300 ms). The nAChR-induced inhibition of DA release does not require depletion of the DA pool but limits axonal Ca²⁺ summation, is detected in vivo and impacts on DA-dependent learning.

### Inhibition Versus Enhancement of DA Release

The Inventors reveal that activation of Chis limits successive DA release by preventing DA axon depolarisation events and axonal Ca²⁺ summation following nAChR activation, and predict from a computational model that tonically active Chis inhibit DA release continuously in vivo. ChIs have been suggested previously to enhance DA release in response to both synchronised excitation and pausing during multiphasic activity^{5-7, 11, 18} Striatal ChIs can directly drive DA release after optogenetic or electrical activation of ChIs or their cortical or thalamic inputs^{5, 6, 10, 20, 41} leading to speculations that initial excitation or rebound activity in multiphasic ChI activity in vivo might drive DA release. However, the observations ex vivo and in vivo now suggest that the levels of excitation and rebound during multiphasic activity in Chis in vivo are insufficient to drive DA axonal release, but rather are sufficient to lead to an inhibition of phasic DA release due to the lower level of nAChR activation required to inhibit than drive DA release. Pauses in a ChI multiphasic response have previously been speculated to promote coincident burst-evoked DA^{7, 18}, because high-frequency train stimulation of both ChIs and DA axons can induce more DA release when nAChRs are turned off. The new finding indicates that elevated levels of DA release arise from a disinhibition; the removal of ChI-induced inhibition. The current finding that ChIs primarily inhibit DA release also suggests that ChI activity driven by excitatory inputs, e.g. from cortex and thalamus, and resultant activation of nAChRs on DA axons^{41, 42}, might inhibit striatal DA release in vivo, and vice versa, inhibitory inputs to ChIs^{43, 44} might boost DA release.

At longer timecourses corresponding to the rebound activity seen in ChI multiphasic activity (100-300 ms after initial activity), it was found that DA release better reflects the stimulation received, consistent with nAChRs being unavailable^{7, 11}. This very likely corresponds to nAChR desensitisation, and suggests that nAChRs activated during initial excitation do not gate DA release at a rebound phase.

The rapid inhibition of DA release following nAChR activation correlated with limited depolarisation in response to subsequent activity, and with consequently limited axonal calcium summation. Prior DA release was not required, together suggesting a rapid ionic mechanism that can occur at levels of axonal activity that are subthreshold for DA release and for action potentials. An ionic mechanism tallies with emerging data indicating that DA output is strongly gated by mechanisms regulating axonal excitability, including DAT activity, K⁺ dependent excitability, and shunting inhibition and Nat-channel inactivation following GABA_{A}-receptor activation^{21, 45}. The differences in strength and duration of ChI-induced inhibition of DA release in DLS versus NAc are accompanied by heterogeneity in nAChR subtypes and consequently channel properties (α4α532* and α4α6β2β3* respectively)^{34, 35}.

### Consequences for ChI Activity on DA Release and Function

Understanding of dopamine function to date is dominated by insights into action potential activity recorded in DA cell bodies. However, by limiting a subsequent depolarization event in DA axons, ChIs might prevent action potential propagation, and play an important role in governing DA function. The very rapid onset of ChI-induced inhibition of DA release (<7 ms) and decay of this mechanisms over ˜50-100 ms could rapidly change DA release inversely in response to dynamic changes in Chis activity without changes in DA neuron activity. This local modulation might be the cause of the extreme case reported recently that DA release increases when no obvious change of DA neuron activity was observed⁸.

For burst activity in DA neurons, this model indicates that the combined effects of the concurrent multiphasic ChI activity principally inhibits DA output, particularly with initial excitation, as a result of nAChR activation. These effects might contribute critically to striatal learning⁴⁶, to changing DA signals after learning²⁹. In addition, the variation seen across striatum in the power and duration of ChI-induced inhibition, stronger in DLS than in NAc, might in turn contribute to diverse DA signals and regulatory mechanisms between regions⁴⁷⁻⁴⁹.

Local antagonism of nAChRs modified the reward-related behaviour of animals, and also successfully remediated motor deficits in 6-OHDA-lesioned mice, indicating that manipulating nAChRs on dopamine axons could be a promising treatment for PD and other dopamine-related diseases. ChI-induced inhibition on DA release, together with the established inhibitory DA-D₂ receptor inhibition of ChIs⁵⁰⁻⁵², provide mechanistic explanations for the long-suspected balance of reciprocal inhibition between striatal ACh and DA. This feedforward loop will amplify any deviation from a balance in the ACh or DA systems. For example, deficits of DA in PD, by disinhibiting ChIs, would exaggerate ChI-induced inhibition of DA release, and lead to a maladaptive further reduction in DA output. Antagonists of nAChRs would mitigate against this maladaptive inhibition of DA release. Furthermore, the desensitisation of striatal nAChRs by nicotine in smokers^{7, 53} would also be expected to remove nAChR-inhibition of striatal DA release, and thereby promote DA-dependent behaviour (alongside mechanisms operating at level of DA neurons⁵⁴).

In summary, the inventors add rapid inhibition to the portfolio of mechanisms through which striatal nAChRs gate DA release, and find that this mechanism dominates as the means through which nAChRs control DA output.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCES

1. Schultz, W., Dayan, P. & Montague, P.R. A neural substrate of prediction and reward. Science 275, 1593-1599 (1997).
2. Hamid, A. A., et al. Mesolimbic dopamine signals the value of work. Nat Neurosci 19, 117-126 (2016).
3. Wickens, J. R. Synaptic plasticity in the basal ganglia. Behav Brain Res 199, 119-128 (2009).
4. Wise, R. A. Dopamine, learning and motivation. Nat Rev Neurosci 5, 483-494 (2004).
5. Cachope, R., et al. Selective activation of cholinergic interneurons enhances accumbal phasic dopamine release: setting the tone for reward processing. Cell Rep 2, 33-41 (2012).
6. Threlfell, S., et al. Striatal dopamine release is triggered by synchronized activity in cholinergic interneurons. Neuron 75, 58-64 (2012).
7. Rice, M. E. & Cragg, S. Nicotine amplifies reward-related dopamine signals in striatum. Nat Neurosci 7, 583-584 (2004).
8. Mohebi, A., et al. Dissociable dopamine dynamics for learning and motivation. Nature 570, 65-70 (2019).
9. Howe, M. W., Tierney, P. L., Sandberg, S. G., Phillips, P. E. & Graybiel, A. M. Prolonged dopamine signalling in striatum signals proximity and value of distant rewards. Nature 500, 575-579 (2013).
10. Wang, L., et al. Modulation of dopamine release in the striatum by physiologically relevant levels of nicotine. Nat Commun 5, 3925 (2014).
11. Zhang, H. & Sulzer, D. Frequency-dependent modulation of dopamine release by nicotine. Nat Neurosci 7, 581-582 (2004).
12. Howe, M., et al. Coordination of rapid cholinergic and dopaminergic signaling in striatum during spontaneous movement. Elife 8 (2019).
13. Rehani, R., et al. Activity Patterns in the Neuropil of Striatal Cholinergic Interneurons in Freely Moving Mice Represent Their Collective Spiking Dynamics. eNeuro 6, ENEURO.0351-0318.2018 (2019).
14. Aosaki, T., Kimura, M. & Graybiel, A.M. Temporal and spatial characteristics of tonically active neurons of the primate's striatum. J Neurophysiol 73, 1234-1252 (1995).
15. Apicella, P., Ravel, S., Deffains, M. & Legallet, E. The role of striatal tonically active neurons in reward prediction error signaling during instrumental task performance. J Neurosci 31, 1507-1515 (2011).
16. Morris, G., Arkadir, D., Nevet, A., Vaadia, E. & Bergman, H. Coincident but distinct messages of midbrain dopamine and striatal tonically active neurons. Neuron 43, 133-143 (2004).
17. Joshua, M., Adler, A., Mitelman, R., Vaadia, E. & Bergman, H. Midbrain dopaminergic neurons and striatal cholinergic interneurons encode the difference between reward and aversive events at different epochs of probabilistic classical conditioning trials. J Neurosci 28, 11673-11684 (2008).
18. Cragg, S. Meaningful silences: how dopamine listens to the ACh pause. Trends Neurosci 29, 125-131 (2006).
19. Patel, J. C., Rossignol, E., Rice, M. E. & Machold, R. P. Opposing regulation of dopaminergic activity and exploratory motor behavior by forebrain and brainstem cholinergic circuits. Nat Commun 3, 1172 (2012).
20. Wang, L., et al. Temporal components of cholinergic terminal to dopaminergic terminal transmission in dorsal striatum slices of mice. J Physiol 592, 3559-3576 (2014).
21. Condon, M. D., et al. Plasticity in striatal dopamine release is governed by release-independent depression and the dopamine transporter. Nature Communications 10, 4263 (2019).
22. Lozovaya, N., et al. GABAergic inhibition in dual-transmission cholinergic and GABAergic striatal interneurons is abolished in Parkinson disease. Nature communications 9, 1422 (2018).
23. Tritsch, N. X., Ding, J. B. & Sabatini, B. L. Dopaminergic neurons inhibit striatal output through non-canonical release of GABA. Nature 490, 262-266 (2012).
24. Lopes, E. F., Roberts, B. M., Siddorn, R. E., Clements, M. A. & Cragg, S. Inhibition of Nigrostriatal Dopamine Release by Striatal GABAA and GABAB Receptors. J Neurosci 39, 1058-1065 (2019).
25. Brimblecombe, K. R., et al. Calbindin-D28K Limits Dopamine Release in Ventral but Not Dorsal Striatum by Regulating Ca2+ Availability and Dopamine Transporter Function. ACS chemical neuroscience 10, 3419-3426 (2019).
26. Brimblecombe, R. K., Gracie, J. C., Platt, J. N. & Cragg, J. S. Gating of dopamine transmission by calcium and axonal N-, Q-, T- and L-type voltage-gated calcium channels differs between striatal domains. The Journal of Physiology 593, 929-946 (2015).
27. Threlfell, S., et al. Striatal muscarinic receptors promote activity dependence of dopamine transmission via distinct receptor subtypes on cholinergic interneurons in ventral versus dorsal striatum. J Neurosci 30, 3398-3408 (2010).
28. Villette, V., et al. Ultrafast Two-Photon Imaging of a High-Gain Voltage Indicator in Awake Behaving Mice. Cell 179, 1590-1608 e1523 (2019).
29. Aosaki, T., et al. Responses of tonically active neurons in the primate's striatum undergo systematic changes during behavioral sensorimotor conditioning. J Neurosci 14, 3969-3984 (1994).
30. Dani, J. A. & Bertrand, D. Nicotinic acetylcholine receptors and nicotinic cholinergic mechanisms of the central nervous system. Annu Rev Pharmacol Toxicol 47, 699-729 (2007).
31. Changeux, J. P., Devillers-Thiery, A. & Chemouilli, P. Acetylcholine receptor: an allosteric protein. Science 225, 1335-1345 (1984).
32. Giniatullin, R., Nistri, A. & Yakel, J. L. Desensitization of nicotinic ACh receptors: shaping cholinergic signaling. Trends Neurosci 28, 371-378 (2005).
33 Shin, J. H., Adrover, M. F. & Alvarez, V. A. Distinctive Modulation of Dopamine Release in the Nucleus Accumbens Shell Mediated by Dopamine and Acetylcholine Receptors. J Neurosci 37, 11166-11180 (2017).
34. Exley, R., et al. Distinct contributions of nicotinic acetylcholine receptor subunit α4 and subunit α6 to the reinforcing effects of nicotine. Proceedings of the National Academy of Sciences 108, 7577-7582 (2011).
35. Exley, R., McIntosh, J. M., Marks, M. J., Maskos, U. & Cragg, S. Striatal alpha5 nicotinic receptor subunit regulates dopamine transmission in dorsal striatum. J Neurosci 32, 2352-2356 (2012).
36. Cunningham, C. L., Gremel, C. M. & Groblewski, P. A. Drug-induced conditioned place preference and aversion in mice. Nat Protoc 1, 1662-1670 (2006).
37. Collins, A. L., et al. Nucleus Accumbens Cholinergic Interneurons Oppose Cue-Motivated Behavior. Biol Psychiat 86, 388-396 (2019).
38. Collins, A. L., Aitken, T. J., Greenfield, V. Y., Ostlund, S. B. & Wassum, K. M. Nucleus Accumbens Acetylcholine Receptors Modulate Dopamine and Motivation. Neuropsychopharmacology 41, 2830-2838 (2016).
39. Qian, H., et al. Reversing a model of Parkinson's disease with in situ converted nigral neurons. Nature 582, 550-556 (2020).
40. Aosaki, T., Graybiel, A. M. & Kimura, M. Effect of the nigrostriatal dopamine system on acquired neural responses in the striatum of behaving monkeys. Science 265, 412-415 (1994).
41. Kosillo, P., Zhang, Y. F., Threlfell, S. & Cragg, S. Cortical Control of Striatal Dopamine Transmission via Striatal Cholinergic Interneurons. Cereb Cortex 26, 4160-4169 (2016).
42. Adrover, M. F., Shin, J. H. & Alvarez, V. A. Glutamate and dopamine transmission from midbrain dopamine neurons share similar release properties but are differentially affected by cocaine. J Neurosci 34, 3183-3192 (2014).
43. Brown, M. T., et al. Ventral tegmental area GABA projections pause accumbal cholinergic interneurons to enhance associative learning. Nature 492, 452-456 (2012).
44. Gonzales, K. K., Pare, J. F., Wichmann, T. & Smith, Y. GABAergic inputs from direct and indirect striatal projection neurons onto cholinergic interneurons in the primate putamen. J Comp Neurol 521, 2502-2522 (2013).
45. Kramer, P. F., Twedell, E. L., Shin, J. H., Zhang, R. & Khaliq, Z. M. Axonal mechanisms mediating gamma-aminobutyric acid receptor type A (GABA-A) inhibition of striatal dopamine release. Elife 9, e55729 (2020).
46. Zhang, Y.-F., Fisher, S. D., Oswald, M., Wickens, J. R. & Reynolds, J. N. J. Coincidence of cholinergic pauses, dopaminergic activation and depolarization drives synaptic plasticity in the striatum. bioRxiv, 803536 (2019).
47. Willuhn, I., Burgeno, L. M., Everitt, B. J. & Phillips, P. E. Hierarchical recruitment of phasic dopamine signaling in the striatum during the progression of cocaine use. Proc Natl Acad Sci USA 109, 20703-20708 (2012).
48. Willuhn, I., Burgeno, L. M., Groblewski, P. A. & Phillips, P. E. Excessive cocaine use results from decreased phasic dopamine signaling in the striatum. Nat Neurosci 17, 704-709 (2014).
49. Hamid, A. A., Frank, M. J. & Moore, C. I. Dopamine waves as a mechanism for spatiotemporal credit assignment. bioRxiv, 729640 (2019).
50. Ding, J. B., Guzman, J. N., Peterson, J. D., Goldberg, J. A. & Surmeier, D. J. Thalamic gating of corticostriatal signaling by cholinergic interneurons. Neuron 67, 294-307 (2010).
51. Augustin, S. M., Chancey, J. H. & Lovinger, D. M. Dual Dopaminergic Regulation of Corticostriatal Plasticity by Cholinergic Interneurons and Indirect Pathway Medium Spiny Neurons. Cell Rep 24, 2883-2893 (2018).
52. Alcantara, A. A., Chen, V., Herring, B. E., Mendenhall, J. M. & Berlanga, M. L. Localization of dopamine D2 receptors on cholinergic interneurons of the dorsal striatum and nucleus accumbens of the rat. Brain Res 986, 22-29 (2003).
53. Zhou, F. M., Liang, Y. & Dani, J. A. Endogenous nicotinic cholinergic activity regulates dopamine release in the striatum. Nat Neurosci 4, 1224-1229 (2001).
54. Faure, P., Tolu, S., Valverde, S. & Naude, J. Role of nicotinic acetylcholine receptors in regulating dopamine neuron activity. Neuroscience 282, 86-100 (2014).
55. Zhang, Y. F., Reynolds, J. N. J. & Cragg, S. Pauses in cholinergic interneuron activity are driven by excitatory input and delayed rectification, with dopamine modulation. Neuron 98, 918-925 e913 (2018).
56. Dajas-Bailador, F. A., et al. Hydroxyl radical production in the substantia nigra after 6-hydroxydopamine and hypoxia-reoxygenation. Brain Res 813, 18-25 (1998).

1. A nicotinic acetylcholine receptor antagonist/blocker, or a pharmaceutically acceptable salt thereof, for use in the treatment of a condition or disorder in which reduced levels of dopamine in the CNS are implicated.

2. The nicotinic acetylcholine receptor (nAChR) antagonist/blocker, for the use according to claim 1, wherein administration of the nicotinic acetylcholine receptor antagonist/blocker promotes dopamine release in the CNS.

3. The nicotinic acetylcholine receptor (nAChR) antagonist/blocker, for the use according to claim 1 or claim 2, wherein administration of the nicotinic acetylcholine receptor antagonist/blocker promotes dopamine release in the brain.

4. A nicotinic acetylcholine receptor (nAChR) antagonist/blocker, for the use according to any one of the preceding claims, wherein administration of the nicotinic acetylcholine receptor antagonist/blocker promotes dopamine release in the striatum.

5. A nicotinic acetylcholine receptor (nAChR) antagonist/blocker, for the use according to any one of the preceding claims, wherein administration of the nicotinic acetylcholine receptor antagonist/blocker promotes dopamine release in the dorsal striatum, ventral striatum, ventral tegmental area, basal ganglia and/or the substantia nigra.

6. A nicotinic acetylcholine receptor (nAChR) antagonist/blocker, for the use according to any one of the preceding claims, wherein the condition or disorder is one in which reduced levels of dopamine in the brain are implicated.

7. A nicotinic acetylcholine receptor (nAChR) antagonist/blocker, for the use according to any one of the preceding claims, wherein the condition or disorder is one in which reduced levels of dopamine in the striatum are implicated.

8. A nicotinic acetylcholine receptor (nAChR) antagonist/blocker, for the use according to any one of the preceding claims, wherein the condition or disorder is one in which reduced levels of dopamine in the dorsal striatum, ventral striatum, ventral tegmental area, basal ganglia and/or the substantia nigra are implicated.

9. A nicotinic acetylcholine receptor (nAChR) antagonist/blocker, for the use according to any one of the preceding claims, wherein the condition or disorder is selected from dopamine deficiency, addiction (including binge eating, nicotine addiction and alcohol addiction), Alzheimer's disease, attention deficit hyperactivity disorder (ADHD), bipolar disorder, dopa-responsive dystonia and DRD-plus, Huntington's disease, multiple sclerosis, obsessive compulsive disorder (OCD), Parkinson's disease (including resting tremor in Parkinson's disease), schizophrenia, tics (i.e. a repetitive involuntary movement or sound), Tourette's syndrome, depression and reward deficiency syndrome.

10. A nicotinic acetylcholine receptor (nAChR) antagonist/blocker, for use according to any one of the preceding claims, wherein the condition or disorder is selected from Parkinson's Disease or ADHD.

11. A nicotinic acetylcholine receptor (nAChR) antagonist/blocker, for use according to any one of the preceding claims, wherein the condition or disorder is Parkinson's Disease.

12. A nicotinic acetylcholine receptor (nAChR) antagonist/blocker, for the use according to any one of the preceding claims, wherein the nicotinic acetylcholine receptor antagonist/blocker is selected from Dihydro-β-erythroidine (DHβE), mecamylamine, bPiDDP (1,1′-(1, 12-Dodecanediyl)bis[3-methylpyridinium]dibromide), methyllycaconitine, MG 624 (N,N, N-Triethyl-2-[4-(2-phenylethenyl)phenoxy]ethanaminium iodide), SR 16584 (1,3-Dihydro-1-(3-exo)-9-methyl-9-azabicyclo[3.3.1]non-3-yl]-2H-indol-2-one), Catestatin, Chlorisondamine diiodide, 2,2,6,6-tetramethylpiperidin-4-yl heptanoate (TMPH), Chlorisondamine, α-conotoxins (e.g. ACV 1, AulB, El, Iml, MII, PIA, PnlA), A 85380 (3-[(2S)-2-Azetidinylmethoxy]-pyridine, ABT 089 (2-Methyl-3-[(2S)-pyrrolidinylmethoxy]pyridine), ABT 594 ((R)-5-(Azetidin-2-ylmethoxy)-2-chloropyridine), Dianicline ((5aS,8S,10aR)-5a,6,9,10-Tetrahydro-7H, 11H-8,10a-methanopyrido[2′,3′:5,6]pyrano[2,3-d]azepine), RJR 2403 ((E)-N-Methyl-4-(3-pyridinyl)-3-buten-1-amine), TC 2559 (4-(5-ethoxy-3-pyridinyl)-N-methyl-(3E)-3-buten-1-amine), Varenicline (7,8,9,10-Tetrahydro-6,10-methano-6H-pyrazino[2,3-h][3]benzazepine), hexamethonium bromide, tubocurarine chloride, α-bungarotoxin, COG 133, D-amphetamine sulfate, PAMP-20, or a pharmaceutically acceptable salt or derivative thereof;
optionally wherein the nicotinic acetylcholine receptor antagonist/blocker is selected from Dihydro-β-erythroidine (DhBE), mecamylamine, bPiDDP, methyllycaconitine, MG 624, SR 16584, Catestatin, Chlorisondamine diiodide, 2,2,6,6-tetramethylpiperidin-4-yl heptanoate (TMPH), Chlorisondamine, α-conotoxins (e.g. ACV 1, AulB, El, Iml, MII, PIA, PnlA), A 85380 dihydrochloride (3-[(2S)-2-Azetidinylmethoxy]-pyridine dihydrochloride, ABT 089 (2-Methyl-3-[(2S)-pyrrolidinylmethoxy]pyridine), ABT 594 ((R)-5-(Azetidin-2-ylmethoxy)-2-chloropyridine), Dianicline ((5aS,8S,10aR)-5a,6,9,10-Tetrahydro-7H, 11H-8,10a-methanopyrido[2′,3′:5,6]pyrano[2,3-d]azepine), RJR 2403 ((E)-N-Methyl-4-(3-pyridinyl)-3-buten-1-amine), TC 2559 (4-(5-ethoxy-3-pyridinyl)-N-methyl-(3E)-3-buten-1-amine), Varenicline (7,8,9,10-Tetrahydro-6,10-methano-6H-pyrazino[2,3-h][3]benzazepine tartrate), or a pharmaceutically acceptable salt or derivative thereof.

13. A nicotinic acetylcholine receptor (nAChR) antagonist/blocker, for the use according to any one of the preceding claims, wherein the nicotinic acetylcholine receptor antagonist/blocker is selected from Dihydro-β-erythroidine (DhBE), mecamylamine, bPiDDP, methyllycaconitine, MG 624, SR 16584, Catestatin, Chlorisondamine diiodide, 2,2,6,6-tetramethylpiperidin-4-yl heptanoate (TMPH), Chlorisondamine and α-conotoxins (e.g. ACV 1, AulB, El, Iml, MII, PIA, PnlA), or a pharmaceutically acceptable salt or derivate thereof.

14. A nicotinic acetylcholine receptor (nAChR) antagonist/blocker for the use according to any one of the preceding claims, wherein the nicotinic acetylcholine receptor antagonist/blocker is selected from Dihydro-β-erythroidine (DhBE), mecamylamine, Chlorisondamine diiodide, 2,2,6,6-tetramethylpiperidin-4-yl heptanoate (TMPH), Chlorisondamine, or a pharmaceutically acceptable salt or derivate thereof.

15. A nicotinic acetylcholine receptor (nAChR) antagonist/blocker, for the use according to any one of the preceding claims, wherein the nicotinic acetylcholine receptor is selected from DHβE and mecamylamine.

16. A nicotinic acetylcholine receptor (nAChR) antagonist/blocker for the use according to any one of the preceding claims, wherein the nicotinic acetylcholine receptor antagonist/blocker is selective for the α4β2 receptor.

17. A nicotinic acetylcholine receptor (nAChR) antagonist/blocker, for the use according to claim 16, wherein the nicotinic acetylcholine receptor antagonist/blocker selective for the α4β2 receptor is selected from A 85380 (3-[(2S)-2-Azetidinylmethoxy]-pyridine dihydrochloride, ABT 089 (2-Methyl-3-[(2S)-pyrrolidinylmethoxy]pyridine), ABT 594 ((R)-5-(Azetidin-2-ylmethoxy)-2-chloropyridine), Dianicline ((5aS,8S,10aR)-5a,6,9,10-Tetrahydro-7H,11H-8,10a-methanopyrido[2′,3′:5,6]pyrano[2,3-d]azepine), RJR 2403 ((E)-N-Methyl-4-(3-pyridinyl)-3-buten-1-amine), TC 2559 (4-(5-ethoxy-3-pyridinyl)-N-methyl-(3E)-3-buten-1-amine) and Varenicline (7,8,9,10-Tetrahydro-6,10-methano-6H-pyrazino[2,3-h][3]benzazepine tartrate), or a pharmaceutically acceptable salt or derivate thereof.

18. A nicotinic acetylcholine receptor (nAChR) antagonist/blocker, for the use according to any one the preceding claims, wherein the nicotinic acetylcholine receptor (nAChR) antagonist/blocker is administered orally, nasally, intraperitoneally, intracerebroventricularly, intrathecally, intracranially, subcutaneously, or intravenously.

19. A nicotinic acetylcholine receptor (nAChR) antagonist/blocker, for the use according to any one the preceding claims, wherein the nicotinic acetylcholine receptor (nAChR) antagonist/blocker is administered directly to the CNS.

20. A nicotinic acetylcholine receptor (nAChR) antagonist/blocker, for the use according to any one the preceding claims, wherein the nicotinic acetylcholine receptor (nAChR) antagonist/blocker is administered via intraperitoneal injection, intravenous injection, local infusion to the CNS or direct injection to the CNS.

21. A nicotinic acetylcholine receptor (nAChR) antagonist/blocker, for use in increasing the level of dopamine in the CNS.

22. The use of a nicotinic acetylcholine receptor (nAChR) antagonist/blocker, or a pharmaceutically acceptable salt thereof, in increasing the level of dopamine in the CNS.

23. A method of treating a condition or disorder in which reduced levels of dopamine in the CNS are implicated, said method comprising administering to a subject in need thereof a therapeutically effective amount of a nicotinic acetylcholine receptor (nAChR) antagonist/blocker or a pharmaceutically acceptable salt thereof.

24. A method of increasing the level of dopamine in the CNS of a subject, the method comprising administering a therapeutically effective amount nicotinic acetylcholine receptor (nAChR) antagonist/blocker to the subject.

25. A pharmaceutical composition which comprises a nicotinic acetylcholine receptor antagonist/blocker, or a pharmaceutically acceptable salt thereof, and one or more pharmaceutically acceptable excipients, for use in the treatment of a condition or disorder in which reduced levels of dopamine in the CNS are implicated.
